(19) United States
(12) Patent Application Publication
(10) Pub. No.: US 2006/0282107 A1
(43) Pub. Date: Dec. 14, 2006

(54) Intragastric device for treating obesity

(76) Inventors: Kiyoshi Hashiba, Sao Paulo (BR); Brian K. Jones, Winston-Salem, NC (US); Kenneth C. Kennedy, Clemmons, NC (US); Vihar C. Surti, Winston-Salem, NC (US)

Correspondence Address: BRINKS HOFER GILSON & LIONE, P.O. BOX 10395, CHICAGO, IL, 60610, US

(21) Appl. No.: 11/430,517

(22) Filed: May 8, 2006

Related U.S. Application Data
(60) Provisional application No. 60/679,135, filed on May 9, 2005.

(51) Int. Cl. A61B 17/08

(52) U.S. Cl. 606/153

(57) ABSTRACT

An apparatus comprising an intragastric member re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, wherein said intragastric member comprises a plurality of spaced apart openings. The intragastric member comprises an elongate member having a proximal end and a distal end, wherein the elongate member is threaded through the openings of the intragastric member, and the intragastric member is disposed between the proximal end and the distal end of the elongate member. The apparatus also comprises a proximal stopper engaged to the distal end of the elongate member and a proximal stopper engaged to the proximal end of the elongate member for securing the intragastric member upon delivery into the gastric lumen of the patient. The elongate member can also comprise a cinching member to facilitate delivery of the intragastric member into the gastric lumen of the patient.

### RELATED APPLICATIONS

This application claims priority to provisional application no. 60/679,135 filed on May 9, 2005, the entire disclosure of which is incorporated by reference herein.

### TECHNICAL FIELD

This invention relates to medical devices, and more particularly to obesity treatment devices that can be placed in the stomach of a patient to reduce the size of the stomach reservoir.

### BACKGROUND OF THE INVENTION

It is well known that obesity is a very difficult condition to treat. Methods of treatment are varied, and include drugs, behavior therapy, and physical exercise, or often a combinational approach involving two or more of these methods. Unfortunately, results are seldom long term, with many patients eventually returning to their original weight over time. For that reason, obesity, particularly morbid obesity, is often considered an incurable condition. More invasive approaches have been available which have yielded good results in many patients. These include surgical options such as bypass operations or gastroplasty. However, these procedures carry high risks, and are therefore not appropriate for most patients.

In the early 1980s, physicians began to experiment with the placement of intragastric balloons to reduce the size of the stomach reservoir, and consequently its capacity for food. Once deployed in the stomach, the balloon helps to trigger a sensation of fullness and a decreased feeling of hunger. These balloons are typically cylindrical or pear-shaped, generally range in size from 200-500 ml or more, are made of an elastomer such as silicone, polyurethane, or latex, and are filled with air, water, or saline. While some studies demonstrated modest weight loss, the effects of these balloons often diminished after three or four weeks, possibly due to the gradual distension of the stomach or the fact that the body adjusted to the presence of the balloon. Other balloons include a tube exiting the nasal passage that allows the balloon to be periodically deflated and re-insufflated to better simulate normal food intake. However, the disadvantages of having an inflation tube exiting the nose are obvious.

The experience with balloons as a method of treating obesity has provided uncertain results, and has been frequently disappointing. Some trials failed to show significant weight loss over a placebo, or were ineffective unless the balloon placement procedure was combined with a low-calorie diet. Complications have also been observed, such as gastric ulcers, especially with use of fluid-filled balloons, and small bowel obstructions caused by deflated balloons. In addition, there have been documented instances of the balloon blocking off or lodging in the opening to the duodenum, wherein the balloon may act like a ball valve to prevent the stomach contents from emptying into the intestines.

Unrelated to the above-discussed methods for treating obesity, it has been observed that the ingestion of certain indigestible matter, such as fibers, hair, fuzzy materials, etc., can collect in the stomach over time, and eventually form a mass called a bezoar. In some patients, particularly children and the mentally handicapped, bezoars often result from the ingestion of plastic or synthetic materials. In many cases, bezoars can cause indigestion, stomach upset, or vomiting, especially if allowed to grow sufficiently large. It has also been documented that certain individuals having bezoars are subject to weight loss, presumably due to the decrease in the size of the stomach reservoir. Although bezoars may be removed endoscopically, especially in conjunction with a device known as a bezotome or bezotriptor, they, particularly larger ones, often require surgery.

What is needed is an intragastric member that provides the potential weight loss benefits of a bezoar or intragastric balloon without the associated complications. Ideally, such a-device should be well-tolerated by the patient, effective over a long period of time, sizable for individual anatomies, and easy to place and retrieve.

### SUMMARY OF THE INVENTION

The foregoing problems are solved and a technical advance is achieved by an illustrative obesity treatment apparatus comprising at least one intragastric member or artificial bezoar made of a digestive-resistant or substantially indigestible material that is introduced into a gastric lumen of a mammal in a first configuration. The intragastric member or artificial bezoar is typically inserted into the gastric lumen in a partially compacted configuration, whereby it is then manipulated into, or allowed to assume, a second expanded configuration sufficiently large to remain within the reservoir of the stomach during normal activities and not be passed through the pylorus and into the intestines. In animals, the present invention has been found to be effective in achieving weight loss over a several month period, while being easy to place and retrieve. Another advance is that the present invention can be effective at a smaller volume within the stomach than existing intragastric members, such as balloons.

In one aspect of the invention, the obesity treatment apparatus comprises an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, wherein said intragastric member comprises a plurality of spaced apart openings. The apparatus further comprises an elongate member having a proximal end and a distal end wherein the elongate member is threaded through the openings of the intragastric member. The intragastric member is disposed between the proximal end and distal end of the elongate member along a plurality of ribs extending between the proximal end and distal end of the elongate member. A distal stopper, such as a pawl, is engaged to the distal end of the elongate member for securing the intragastric member along the elongate member and a proximal stopper, such as a second pawl, is engaged to the proximal end of the elongate member for locking the intragastric member along the elongate member. The apparatus also comprises a cinching member, such as a nylon thread or similar thread-like structure, having a proximal end and a distal end wherein the proximal end is engaged to a distal end of the proximal stopper and the distal end is engaged to the proximal end of the elongate member. The cinching member is for moving the proximal stopper along the elongate member towards the distal stopper.

The elongate member of the apparatus can include a connector engaged to the proximal end of the elongate member wherein the connector comprises a lumen configured to receive the cinching member as it passes from a lumen of the proximal stopper. A second distal stopper, such as a drag, is engaged to the distal end of the elongate member between the distal stopper and the plurality of ribs while a second proximal stopper, such as a second drag, is engaged along the cinching member.

In yet another embodiment of the invention, the obesity treatment apparatus comprises an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, wherein said intragastric member comprises a plurality of spaced apart openings. The apparatus further comprises an elongate member having a proximal end and a distal end wherein the intragastric member is threaded along a plurality of ribs extending between the proximal end and the distal end of the elongate member. A distal stopper, such as a button, is engaged to the distal end of the elongate member for securing the intragastric member along the elongate member. A proximal stopper, such as a second button, is engaged to the proximal end of the elongate member for locking the intragastric member along the elongate member. The apparatus also comprises a cinching member, such as a nylon thread or similar thread-like structure, having a proximal end and a distal end. The proximal end of the cinching member is engaged to the proximal stopper and extends through a lumen of the elongate member wherein the distal end of the cinching member is engaged to the distal stopper.

The elongate member further includes a locking member engaged along a distal end of the elongate member, wherein the locking member engages the distal stopper upon delivery of the intragastric member into the gastric lumen. The locking member comprises a lumen to receive the cinching member as it passes through the lumen of the elongate member. The proximal stopper also comprises a lumen to receive the cinching member as it passes through the lumen of the elongate member. Additionally, the apparatus includes at least one second distal stopper engaged with the distal end of the elongate member to secure the intragastric member upon delivery into the gastric lumen. When the intragastric member is secured, the distal stopper is engaged to the proximal stopper by pulling the cinching member proximally to remove the cinching member from the lumen of the elongate member. In addition, the proximal stopper is engaged to the plurality of ribs of the elongate member by pulling the cinching member proximally to remove the cinching member from the lumen of the elongate member.

In yet another embodiment of the invention, the obesity treatment apparatus comprises an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus. The apparatus further comprises a cinching member, such as a nylon thread or similar thread-like structure, having a proximal end and a distal end wherein the intragastric member is threaded between the proximal end and the distal end of the cinching member. A distal stopper is engaged to the distal end of the cinching member for securing the intragastric member along the cinching member. A proximal stopper and a second proximal stopper are engaged to the proximal end of the cinching member for locking the intragastric member along the cinching member.

The distal stopper comprises a first lumen and a second lumen to receive the cinching member as it is passes from the proximal stopper. The proximal stopper also comprises a lumen for receiving the cinching member as it passes from the distal stopper. The second proximal stopper comprises a first lumen, a second lumen and a third lumen for receiving the cinching member as it passes from the distal stopper. When the intragastric member is secured, the proximal stopper is engaged to the second proximal stopper by pulling the cinching member proximally through the second lumen and third lumen of the second proximal stopper. In this embodiment, the distal stopper and the proximal stopper can also include a pair of preformed expandable stoppers having an umbrella-like shape.

In another embodiment of the invention, the obesity treatment apparatus comprises an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus. The apparatus further comprises a first cinching member having a proximal end and a distal end wherein the intragastric member is threaded between the proximal end and the distal end of the first cinching member. The apparatus also comprises a second proximal stopper, such as a bead, having a proximal end and a distal end, wherein the proximal end is engaged to a second cinching member and the distal end is engaged to the first cinching member. A distal stopper is engaged to the distal end of the first cinching member for securing the intragastric member along the first cinching member while a proximal stopper is engaged to the proximal end of the first cinching member for locking the intragastric member along the first cinching member. The first cinching member and second cinching member can comprise a nylon thread or similar thread-like structure.

The second proximal stopper further comprises a first lumen for receiving the first cinching member as it passes from the distal stopper and a second lumen for receiving the second cinching member as it passes from the proximal stopper. The distal stopper comprises a first lumen, a second lumen and a third lumen, wherein the first lumen receives the first cinching member as it passes through the second lumen and the third lumen to engage the second proximal stopper. The distal stopper also comprises a fourth lumen for securing a wire guide to the distal stopper to facilitate delivery into the gastric lumen. The proximal stopper comprises a first lumen and a second lumen for receiving the second cinching member as it passes from the second proximal stopper, wherein the first lumen comprises a first diameter and the second lumen comprises a second diameter. The first diameter is smaller than the second diameter for securing the bead member in the first lumen of the proximal stopper. When the intragastric member is secured, the second proximal stopper is engaged to the proximal stopper by pulling the second cinching member proximally through the first lumen of the proximal stopper. In addition, the first diameter allows the second proximal stopper to pass distally through the first lumen and prevents the second proximal stopper from passing proximally through the first lumen of the proximal stopper.

In yet another embodiment of the invention, the obesity treatment apparatus comprises an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus. The apparatus further comprises an outer delivery tube having a main lumen, a proximal end, and a distal end, wherein the intragastric member is loaded between the proximal end and distal end of the outer delivery tube in the first configuration. An inner delivery tube is engaged with the main lumen of the outer delivery tube and at least one stopper is engaged with the inner delivery tube to secure the intragastric member upon delivery into the gastric lumen. The apparatus also includes an overtube comprising a proximal end, a distal end, and a lumen configured to receive the intragastric member in the first configuration for delivery into the gastric lumen wherein the intragastric member is expanded to the second configuration. The overtube comprises a groove extending about and around the surface of the overtube wherein the groove is in communication with a plurality of rollers to facilitate delivery of the intragastric member into the gastric lumen.

In yet another aspect of the invention, the intragastric member can comprise a single strip of material having a series of apertures spaced along the length thereof, wherein the strip of material is bundled into a series of folds by passing a cinching member through the apertures and cinching the strip of material together. The intragastric member is inserted into the gastric lumen by passing the apertures of the strip of material over a wire guide, preferably in separate bundles, until the entire strip has been accumulated and bundled together inside the gastric lumen with a cinching member. The cinching member can be cut to allow the bundles to separate, thereby facilitating its removal by grasping and pulling one end of the strip.

In another embodiment of the invention, the obesity treatment apparatus comprises an intragastric member having a plurality of openings extending along the surface of the intragastric member to reduce the mass of the intragastric member. The intragastric member can also include a folded edge, wherein the folded edge engages a roller mechanism to facilitate the delivery of the intragastric member into the gastric lumen.

In yet another embodiment of the invention, the obesity treatment apparatus comprises a cutting member having a handle portion engaged to a proximal end of the cutting member and a wire portion engaged to a distal end of the cutting member. The wire portion of the cutting member comprises a hook for cutting or removing a suture of an intragastric member. The cutting member is used to facilitate the removal of the intragastric member.

The intragastric member can also include one or more elements selected from the group consisting of plastic, nylon, polyesters, polyurethanes, polyethylenes, polyamides, silicone and biocompatible polymers to which food will generally not adhere. The intragastric member may also comprise a continuous strip of material that has been folded to form a plurality of loops, said plurality of loops being connected together to form a shape suggestive of a butterfly or bow-tie.

These and other advantages, as well as the invention itself, will become apparent in the details of construction and operation as more fully described below. Moreover, it should be appreciated that several aspects of the invention can be used with other types of intragastric devices or procedures used for the treatment of obesity.

### BRIEF DESCRIPTION OF SEVERAL VIEWS OF THE DRAWINGS

Several embodiments of the present invention will now be described by way of example with reference to the accompanying drawings, in which:

FIG. 1 depicts a pictorial view of an intragastric member of the present invention;

FIG. 2 depicts a pictorial view of the embodiment of FIG. 1 with a delivery system;

FIG. 3 depicts a sectional view of the delivery system of FIG. 2;

FIGS. 4-5 depicts a pictorial view of a pair of intragastric members of the present invention prior to, and after being coupled together;

FIGS. 6-7 depict detail views of different embodiments of indigestible members of intragastric members of the present invention;

FIG. 8 depicts a partially sectional side view of an expandable intragastric member of the present invention;

FIG. 9 depicts a pictorial view of an intragastric member of the present invention being delivered from an outer catheter;

FIG. 10 depicts a pictorial view of an intragastric member of the present invention that includes a splittable outer sheath;

FIG. 11 depicts a side view of an intragastric member of the present invention encased in a dissolvable outer package;

FIG. 12 depicts a pictorial view of an intragastric member of the present invention being manipulated by an endoscopic device;

FIG. 13 depicts a set of intragastric members of the present invention bundled together by a coupling mechanism;

FIG. 14 depicts a schematic cross-sectional view taken along line 1414 of FIG. 13;

FIG. 15 depicts a pictorial view of another embodiment of an intragastric member of the present invention;

FIG. 16 depicts a pictorial view of the embodiment of FIG. 15 separated into separate bundles and ready for insertion into the gastric lumen;

FIG. 17 depicts a portion of the strip material that is used to form the embodiment of FIG. 15;

FIG. 18 depicts the insertion of the separate bundles of FIG. 16 being inserted into the gastric lumen;

FIG. 19 depicts a pictorial view of yet another embodiment of an intragastric member of the present invention;

FIG. 20 depicts a pictorial view of the embodiment of FIG. 19 separated into separate bundles and ready for insertion into the gastric lumen;

FIG. 21 depicts a portion of the strip material that is used to form the embodiment of FIG. 19;

FIG. 22 depicts the insertion of the separate bundles of FIG. 20 being inserted into the gastric lumen;

FIG. 23 depicts a pictorial view of yet another embodiment of an intragastric member of the present invention;

FIGS. 24-25 depict an alternative method of inserting an intragastric member of the present invention into the gastric lumen;

FIG. 26 depicts a pictorial view of yet another embodiment of an intragastric member of the present invention;

FIG. 27 depicts a stopper and cinching member used for the insertion of bundles into the gastric lumen;

FIG. 28-29 depicts an inner delivery tube and outer delivery tube used for the insertion of bundles into the gastric lumen;

FIG. 30 depicts digestive-resistant material threaded onto an outer delivery tube for insertion into the gastric lumen;

FIG. 31 depicts a delivery tip of the outer delivery tube of FIG. 30;

FIG. 32-33 depicts the insertion of separate bundles of FIG. 30 into the gastric lumen;

FIG. 34 depicts a portion of the strip material that is used to form the embodiment of FIG. 30;

FIG. 35 depicts a partial, cross-sectional view of a flexible overtube according to an embodiment of the present invention;

FIG. 36 depicts a partial sectional view of a flexible overtube according to an embodiment of the present invention;

FIG. 37 depicts a perspective view of a flexible overtube according to an embodiment of the present invention;

FIG. 38 depicts a rear perspective view of the valve of FIG. 36;

FIG. 39 depicts a front perspective view of the valve of FIG. 36;

FIG. 40 depicts a front side view of the valve of FIG. 36;

FIG. 41 depicts a sectional view taken along line C-C′ of FIG. 40;

FIG. 42 depicts a sectional, perspective view of the flexible overtube of FIG. 36 having a reinforcement member;

FIG. 43 depicts a partial, cross-sectional view showing the flexible overtube of FIG. 35 positioned in the mouth and along the esophagus of a patient such that the overtube distal end is positioned in the gastric lumen of the stomach;

FIGS. 44-45 depicts a pictorial view of yet another embodiment of an intragastric member of the present invention;

FIGS. 46-47 depicts a perspective view of an umbrella stopper of the intragastric member of FIG. 45;

FIG. 48 depicts a perspective view of an overtube having a plurality of rollers along the lumen of the overtube;

FIG. 49 depicts a sectional view of an overtube having a plurality of grooves along the lumen of the overtube;

FIG. 50 depicts a partial sectional view of an overtube having a plurality of grooves along the lumen of the overtube;

FIG. 51 depicts a pictorial view of an overtube having a plurality of rollers along the surface of the overtube for delivery of an intragastric member;

FIG. 52 depicts a pictorial view of a distal tip of the overtube of FIG. 51;

FIG. 53 depicts a pictorial view of another embodiment of a distal tip of the overtube of FIG. 51;

FIG. 54 depicts a pictorial view of a chain and pulley gear mechanism utilized with the overtube of FIG. 51;

FIG. 55 depicts a pictorial view of another embodiment of an overtube having a plurality of rollers along the surface of the overtube for delivery of an intragastric member;

FIG. 56 depicts a pictorial view of an intragastric member of the present invention wherein the intragastric member comprises a folded edge;

FIG. 57 depicts a pictorial view of the intragastric member of FIG. 56 being delivered with a roller mechanism;

FIG. 58 depicts a pictorial view of a cutting member of the present invention;

FIG. 59 depicts a side view of the cutting member of FIG. 58;

FIG. 60 depicts a sectional view of the cutting member of FIG. 58 extended from a sheath;

FIG. 61 depicts a sectional view of the cutting member of FIG. 60 retracted into the sheath;

FIG. 62 depicts a pictorial view of an intragastric member of the present invention wherein the intragastric member comprises a plurality of openings;

FIG. 63 depicts a pictorial view of an embodiment of an elongate member comprising a cinching member of the present invention;

FIG. 64 depicts a pictorial view of the elongate member of FIG. 63 threaded with an intragastric member;

FIG. 65 depicts a pictorial view of the elongate member of FIG. 64 secured to the intragastric member upon delivery into the gastric lumen;

FIG. 66 depicts a pictorial view of another embodiment of an elongate member comprising a cinching member of the present invention;

FIG. 67 depicts a pictorial view of the elongate member of FIG. 66 threaded with an intragastric member;

FIG. 68 depicts a pictorial view of the elongate member of FIG. 67 secured to the intragastric member upon delivery into the gastric lumen;

FIG. 69 depicts a pictorial view of another embodiment of a cinching member of the present invention;

FIG. 70 depicts a pictorial view of the cinching member of FIG. 69 secured to an intragastric member;

FIG. 71 depicts a pictorial view of another embodiment of a first cinching member and a second cinching member comprising and an intragastric member threaded to a delivery device during delivery into the gastric lumen;

FIG. 72 depicts a pictorial view of the first cinching member and the second cinching member of FIG. 71 attached to the intragastric member during delivery into the gastric lumen;

FIG. 73 depicts a pictorial view of the first cinching member and the second cinching member of FIG. 71 attached to a second locking member during delivery into the gastric lumen;

FIG. 74 depicts a pictorial view of the first cinching member of FIG. 71 secured to the intragastric member upon delivery into the gastric lumen;

FIG. 75 depicts a pictorial view of a second proximal stopper engaged to the first cinching member and the second cinching member of FIG. 73;

FIG. 76 depicts a distal view of a distal stopper of FIG. 73

FIG. 77 depicts a proximal view of the distal stopper of FIG. 73;

FIG. 78 depicts a distal view of a proximal stopper of FIG. 73;

FIG. 79 depicts a distal view of the proximal stopper of FIG. 73 wherein the proximal stopper is engaged to the second proximal stopper; and

FIG. 80 depicts a proximal view of the proximal stopper of FIG. 74 wherein the second proximal stopper is secured to the proximal stopper.

### DETAILED DESCRIPTION OF THE INVENTION

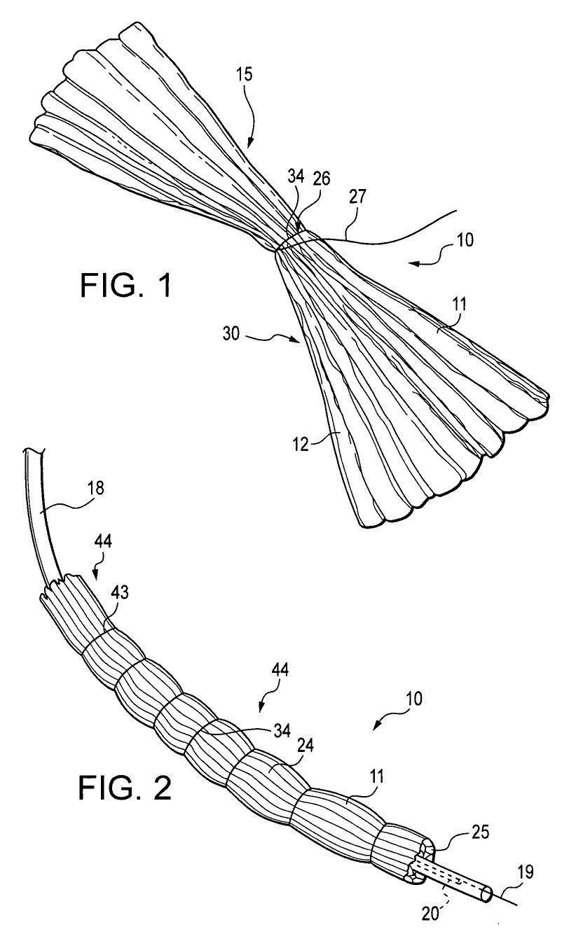

The obesity treatment apparatus 10 of the present invention depicted in FIGS. 1-25 comprises one or more intragastric members 11, each comprising one or more digestive-resistant or indigestible member 12 sized and configured such that the intragastric member 11 can be placed into the stomach of a mammalian patient and reside therein, and being generally unable to pass through the pylorus. As used herein, the terms digestive-resistant and indigestible are intended to mean that the material used is not subject to the degradative effects of stomach acid and enzymes, or the general environment found within the gastric system over an extended period of time, therefore allowing the device to remain intact for the intended life of the device. This does not necessarily mean that the material cannot be degraded over time; however, one skilled in medical arts and gastrological devices would readily appreciate the range of material that would be suitable for use as a long-term intragastric member.

Many well-known plastics have suitable properties, including selected polyesters, polyurethanes, polyethylenes, polyamides, silicone, or other possible materials. Mammalian hair has been found to form natural bezoars, and thus, is also a possible material. However, some materials, such as certain polyamides, have been found to expand over time, which can be an undesirable property. Most other natural materials are generally much less resistant to acids and enzymes, and would therefore typically require treatment or combination with resistant materials to function long term, unless a shorter-term placement is intended or desired.

In the preferred embodiments, the digestive-resistant or indigestible member 12 comprises a low density polyethylene having a thickness of about 40-50 microns. Fluorinated ethylene propylene, ethylene vinyl acetate copolymer, nylon, or types of polymers that are biocompatible and to which food will generally not adhere may also be utilized.

FIG. 1 depicts a single intragastric member 11 in which the digestive-resistant members 12 include a plurality of elongate plastic strips 30 that are secured together in the middle by a retaining element 34, such as a nylon thread. The thread can be elongated to serve as a coupling mechanism 26, such as a tether 27. The number of digestive-resistant members 12 or strips 30 used to form the intragastric member 11 depends on the material used, their length and width, and how many intragastric members 11 comprise a set or grouping. The optimal length of the intragastric member 11 is determined by considering these same factors, as well by what is determined through experimentation to work best.

Feasibility studies have been primarily limited to placement in pigs with both 8 cm and 16 cm intragastric members being used, both having a total volume of about 40 ml when placed in the stomach of the animal. Although the experiments were designed to establish the safety of the device, significant weight loss was nevertheless observed in the test animals. Although no gastric ulcers were found in animals with polyester intragastric members, there was a 20% incidence of gastric ulcers in animals having polyamide devices.

Results from human trials may lead to modifications in the configuration being depicted in the figures of this application. Nevertheless, it is already understood that the dimensions shape, and construction of the intragastric member can be quite variable and still produce the desired results. For example, FIGS. 6-7 depict an alternative digestive-resistant member 12. In the embodiment shown in FIG. 6, the strips 30 of FIG. 1 are replaced by digestive-resistant member 12 comprising a folded or pleated sheet 31 of plastic or other material. Either a single sheet 31 or multiple sheets can be used to form the intragastric member 11 of this embodiment. The embodiment shown in FIG. 7 depicts an intragastric member 11 in which the digestive-resistant members 12 comprise a plurality of elongated fibers or hairs 32, typically made of polymer or other synthetic material.

In the illustrative embodiments, the retaining element 34 (see FIG. 1) is located about the center of the device to hold the digestive-resistant members 12 together. However, a skilled artisan would appreciate that other designs utilizing differently placed retaining elements 34, or eliminating them entirely, could also be utilized. For example, FIG. 8 depicts an expandable device 33 that comprises a retaining element 34 at one end to secure the digestive-resistant members 12, which in this embodiment are typically made of a material having a certain degree of stiffness. The other end is secured by a second, slidable retaining element 41 that is disposed over a tether 27 attached to the first retaining member 34. The intragastric member 11 is deployed in an elongated configuration with the retaining elements 34, 41 located near their maximum possible difference apart. After the device is placed in the gastric lumen, the slidable retaining element 41 is urged along the tether 27 and toward the first retaining element 34 by using a tube, probe, or other device, until the digestive-resistant members 12 have bowed outward, thus increasing the overall dimensions and volume of the device. The slidable retaining element 41 continues to grip the tether 27 after the urging mechanism is removed, retaining the increased dimensions of the intragastric member 11 until further manipulation is needed to reduce its diameter for removal from the patient.

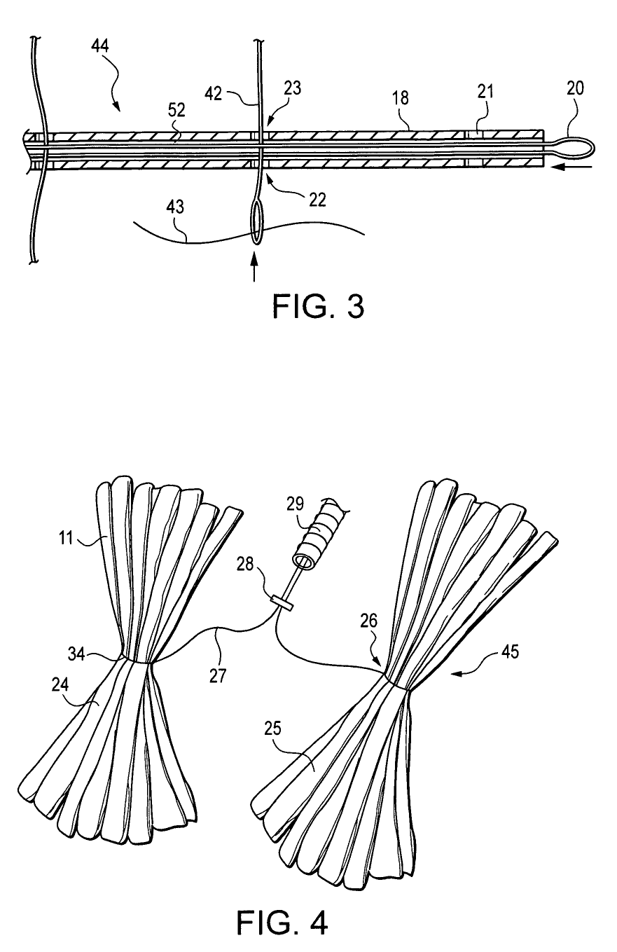

Deployment of intragastric member 11 can be accomplished in a number of ways, depending on the size, number, and configuration of the devices, or according to physician or patient preference. FIGS. 2-4 depict one such delivery system 44 in which first and second intragastric members 24, 25 are mounted over a plastic overtube 18 and secured by a series of suture ties 43, such as cotton thread. A wire guide 19 is typically used in the procedure, and is placed through the passageway 52 of the overtube 18. As shown in FIG. 3, the overtube 18 includes a plurality of apertures 21, a pair of which (e.g., apertures 22 and 23) are distributed approximately every 2 cm along the distal portion of the overtube 18. To secure the intragastric members 24, 25, the suture tie is pulled through the first aperture 22 using a device 42 such as a loop, hook, snare, etc. It is fed through a releasing mechanism 20, such as the illustrative wire loop, and then pulled through the opposite aperture 23. The intragastric members 24, 25 are then placed on the overtube 18, and the suture ties 43 are secured, thereby constraining the intragastric members into a first configuration 14 for delivery. Once the delivery system 44 has been introduced into the gastric lumen, the releasing mechanism 20 is pulled back through the overtube 18, thereby severing the suture ties 43 one by one and releasing the intragastric members 11 into the gastric lumen where they can assume a second configuration 10 (see FIG. 1) that is sufficiently voluminous such that they cannot pass from the stomach.

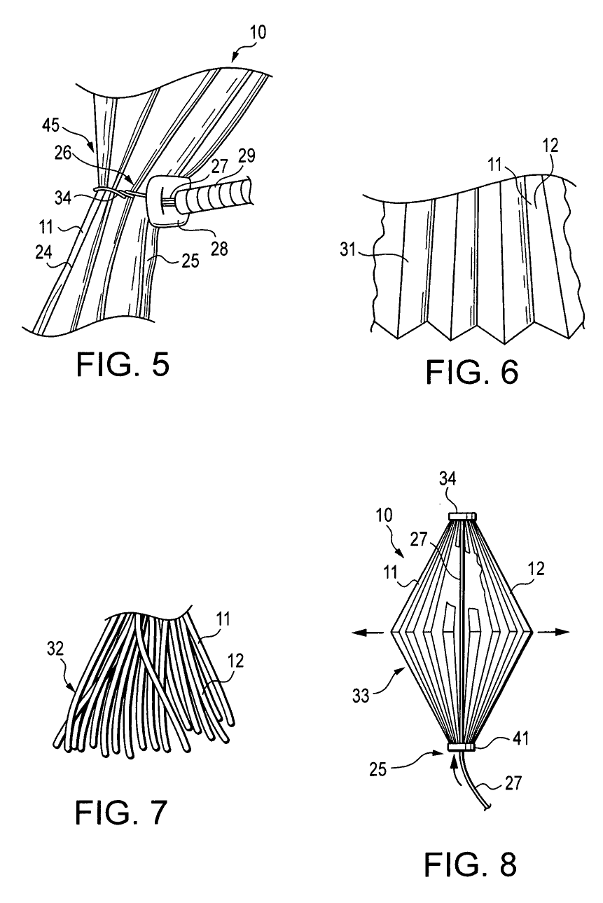

In order to create an obesity treatment apparatus 10 that will be retained in the stomach, it may be necessary that the intragastric members 11 be coupled together to form a grouping or set 45 of intragastric members. FIG. 4 shows the two deployed intragastric members 24, 25 that each have a coupling mechanism 26 (tether 27) attached about them such that they can be drawn together as depicted in FIG. 5. A push member 29, such as a corrugated metal tube, is placed into gastric lumen by using an endoscope, and is guided over the tethers 27 to urge a securing element 28, such as a rubber patch, tightly against the two intragastric members 24, 25. The tethers 27 can then be cut, allowing the grouping 45 to float free within the stomach. This method can also be used to join additional intragastric members 11 to form a larger grouping 45. Likewise, the illustrative delivery system 44 of FIG. 2 can be used to deliver any practical number of intragastric members 11, which can then be joined in the manner described above, or they can be delivered singly or in pairs, and then grouped together after all of the intragastric members 11 have been placed.

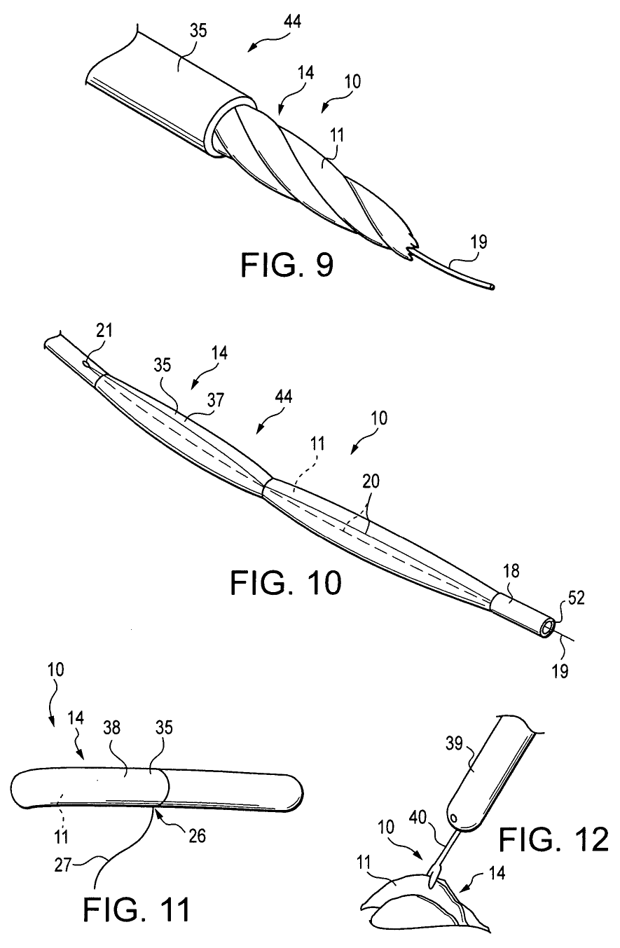

FIGS. 9-11 depict intragastric members 11 that are delivered into the gastric lumen within an outer member 35, such as a sheath, tube, package, wrapping, etc., and subsequently released. For example, FIG. 9 depicts a delivery system 44 in which the intragastric member 11 (or multiple devices) is preloaded into an outer tube or introducer, then deployed therefrom by being pushed out by using a pusher member (not shown). The intragastric member 11 is shown twisted to aid in loading and deployment.

FIG. 10 depicts a delivery system 44 in which the intragastric member is loaded over a tube 18 (as in FIG. 2), but is secured by an outer member 35 comprising a splittable sheath 37 or sleeve made of a thin plastic material. In the illustrative embodiment, the releasing mechanism 20 comprises a nylon thread or wire that is looped under and over the sheath 37, such that it can be withdrawn to tear through the thin material of the sheath 37 to release the intragastric member(s) 11 mounted on the tube 18. The releasing mechanism of FIG. 10 feeds into an aperture 21 and passageway 52 of the tube 18, where it extends to the proximal end of the apparatus 10. Other types of splittable sheaths 37 can also be used, such as the COOK® PEEL-AWAY Introducer Sheath.

FIG. 11 depicts an intragastric member 11 that includes an outer member 35 comprising a dissolvable enclosure 38. The material, such as cellulose, gelatin, or some other dissolvable or rapidly degrading synthetic or biomaterial material, allows the intragastric member 11 to be deployed in the first configuration 14 into the stomach, where it expands into the second configuration 15 (see, e.g., FIG. 1) once the outer enclosure 38 has dissolved or degraded away. The embodiment of FIG. 11 can be delivered with or without a catheter-based delivery system 44, or swallowed by the patient, depending on the outer dimensions of the apparatus 10.

FIG. 12 also depicts a method of delivering the apparatus 10 of the present invention without a catheter or tube 18. It has been found that the intragastric members 11 can be pulled into the gastric lumen using an endoscope 39 and endoscopic instrument 40, such as a forceps, basket, snare, etc. This technique can be employed to pull groupings 45 (see, e.g., FIG. 4) of intragastric members 11 into the gastric lumen, as long as the alimentary tract is sufficiently wide to accommodate the grouping 45.

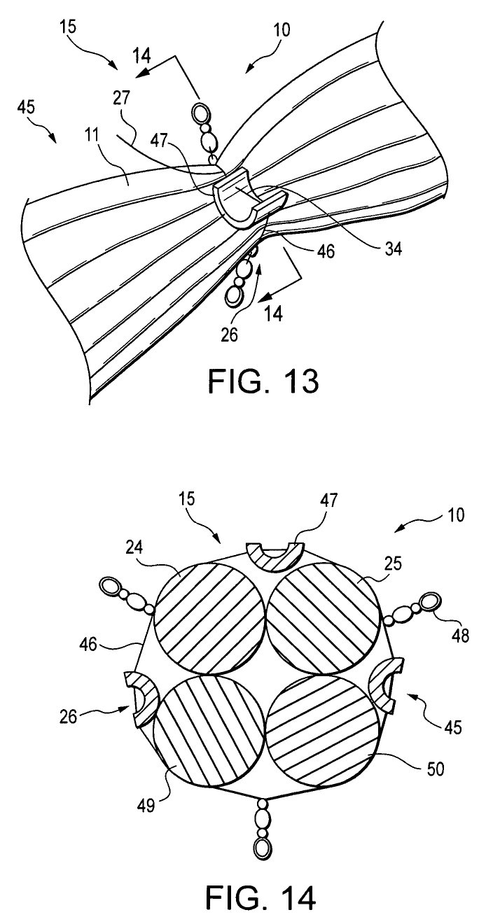

FIGS. 13-14 depict a grouping 45 of four intragastric members 24, 25, 49, 50 that are pre-coupled to one another by a coupling mechanism 26 prior to introduction into the gastric lumen. Although such an arrangement or grouping 45 is sufficiently small such that it can be introduced into the gastric lumen as a set, the adherence of mucous and other changes that occur within the stomach environment can, over time, significantly increase the volume of the apparatus 10 from, for example, an original size of about 60 ml up to a possible size of about 150 ml. The increased size can make it very difficult to remove the grouping 45 from the stomach. To address this problem, multiple intragastric members 45 are grouped together for introduction, and then cut apart when it is time to remove them from the patient. The coupling mechanism 26 comprises a grouping mechanism 46, such as a nylon thread (e.g., standard nylon fishing line), that is wrapped around the grouping 45 to pull them into close contact with one another. The grouping is released by severing the line comprising the grouping mechanism 46 and the intragastric members 24, 25, 49, 50 are removed one at time using a retrieval device such as that shown in FIG. 12.

To assist the operator in cutting the line 46 to release the grouping 45, two different coupling components 47, 48 are included in the illustrative embodiment. The first coupling component 47 comprises a curved polymer piece which is traversed by the line 46 in such a manner that the line 46 can be readily visualized under the scope, thereby providing a place to grasp and/or cut the line with an instrument extending from the endoscope. The second coupling component 48 comprises a fishing line swivel, which being metal, can be readily visualized, as well as providing a hard surface against which a cutting device can be applied to sever the line 46, especially if the line has proved difficult to cut using other methods. It also provides an easily accessible point on the apparatus 10 which can be grabbed with a forceps or other device.

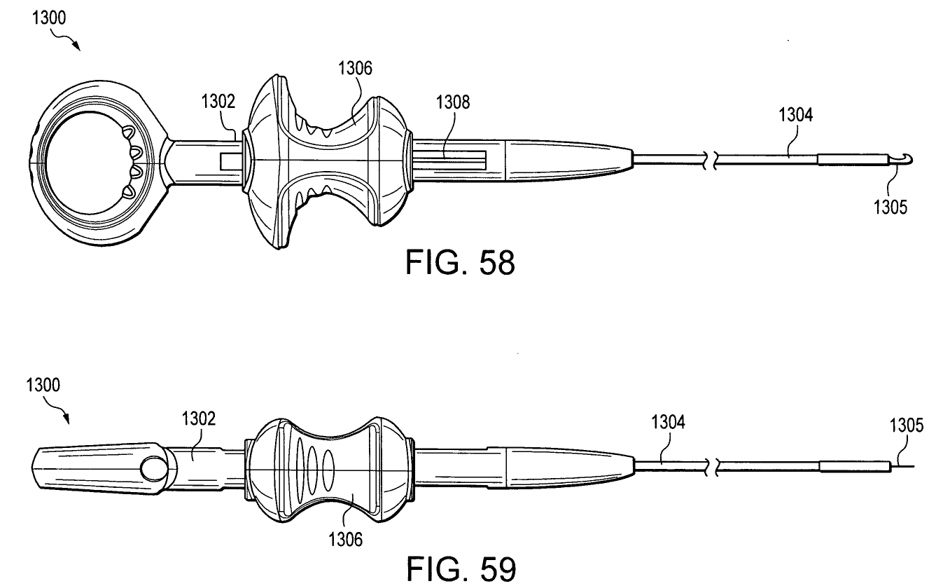

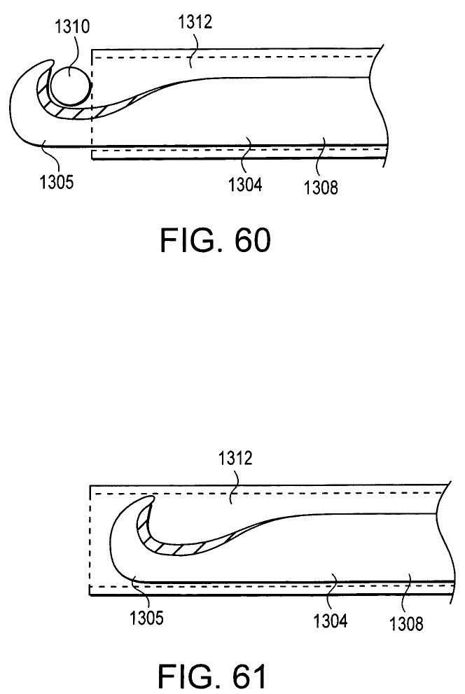

For example, FIGS. 58-61 depict a cutting device 1300 of the present invention. The cutting device 1300 is utilized for cutting and removing the intragastric member after insertion into the gastric lumen of the patient. The cutting device 1300 comprises a proximal end 1302 and a distal end 1304, wherein a handle member 1306 extends along the proximal end 1302 and a wire portion 1308 extends along the distal end 1304 of the cutting device 1300. The wire portion 1308 comprises a hook 1305 engaged to the distal end of the wire portion 1308. The hook 1305 is utilized to remove a suture 1310 or tubing of the intragastric member. The cutting device 1300 can be inserted into a sheath 1312 and delivered through an endoscope into the gastric lumen. During use, the hook 1305 of the cutting device 1300 is extended from the sheath 1312 positioned about the suture 1310, and then is retracted, which results in the cutting of the suture 1310 (FIGS. 60-61). The sheath 1312 provides a protective layer to secure the hook 1305 from damaging the lumen of the endoscope or other delivery device.

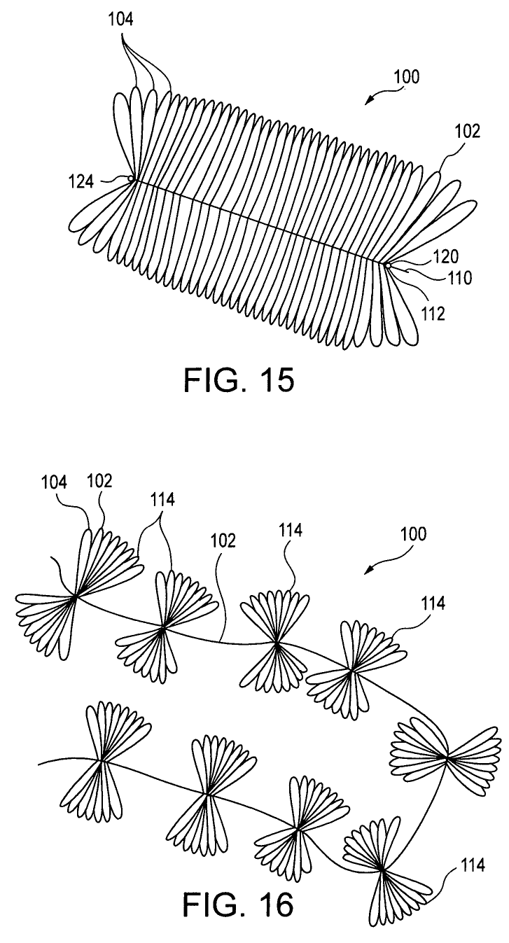

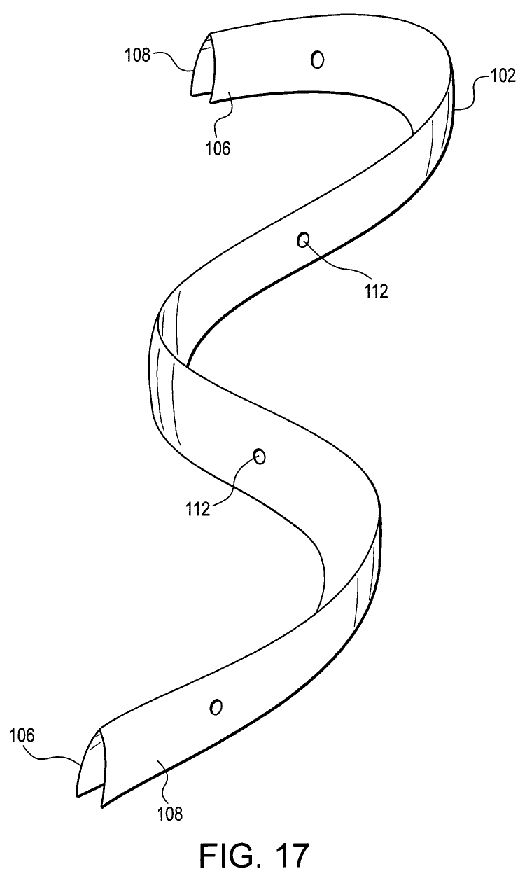

FIG. 15 depicts another embodiment of an intragastric member 100 of the present invention. In this embodiment, the intragastric member 100 comprises a single strip of high-density polyethylene 102 that has been folded and bundled to form eighty-nine (89) loops 104 in the general shape of a butterfly or bow-tie. As best seen in FIG. 17, the single strip of high-density polyethylene 102 of the embodiment is formed from a tube of material having a wall thickness of 40-50 microns and a perimeter of 3 cm that has been sliced in half. Each half of the material is then folded to form a strip 102 having two walls 106,108, wherein each wall 106,108 has a width of 1.5 cm. Of course, the strip 102 could comprise a different number of walls 106, 108, have a different width and thickness, or be formed from a tube of material.

In the embodiment of the intragastric member 100 shown in FIG. 15, each loop 104 is 20 cm in length. Accordingly, the intragastric member 100 is formed from single strip 102 having a total length of approximately 35.6 m.

The intragastric member 100 is bundled by passing a cinching member 110 through an aperture 112 in the strip 102 at the center of the each loop 104. In this embodiment, the cinching member 110 comprises a nylon thread or similar thread-like structure. As best seen in FIG. 17, the apertures 112 are formed in each wall 106, 108 of the strip 102, and are spaced so that loops 104 are formed 20 cm in length when adjacent apertures 112 are pulled together to form the intragastric member 100 shown in FIG. 15. In other words, the apertures 112 are located every 20 cm along the length of the strip 102.

The embodiment of the intragastric member 100 shown in FIG. 15 may be too large for delivery or insertion into the gastric lumen while in its bundled, final configuration. Accordingly, the intragastric member 100 is preferably inserted into the gastric lumen in stages. For example, and as shown in FIG. 16, the intragastric member 100 is separated into nine (9) separate bundles 114, each of which comprise approximately ten (10) loops 104 of the strip 102. The loops 104 of each separate bundle 114 are temporarily grouped or held together by a twist tie 116 or similar device. Grouping the separate bundles 114 in this manner improves the handling of the material and prevents the strip 102 from becoming tangled or contaminated.

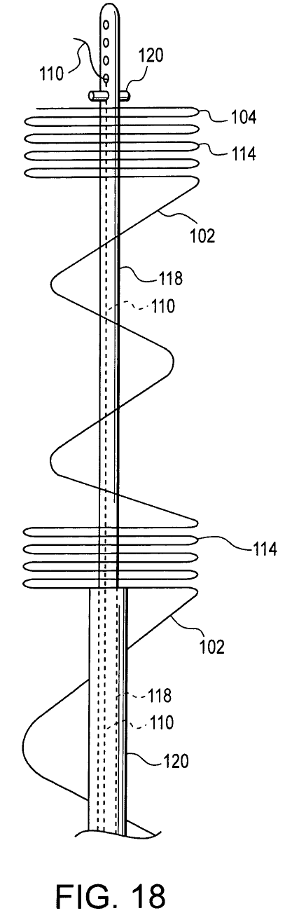

As shown in FIG. 18, the separate bundles 114 of the intragastric member 100 are inserted into the gastric lumen one at a time by using a wire guide 118 such as a Savary-Gilliard™ wire guide, manufactured by Cook Endoscopy, Winston-Salem, N.C. The wire guide 118 comprises a central opening through which the cinching member 110 passes. The end of the cinching member 110 is connected to or tied around a small piece of nylon tubing 120 that is sized so as to not pass through the apertures 112 in the strip 102. Prior to the insertion procedure, the nylon tubing 120 is placed near the distal (forward or insertion) end of the wire guide 118 so as to prevent the strip 102 of the first bundle 114 from sliding off the end of the wire guide 118.

Once the distal end of the wire guide 118 is positioned in the gastric lumen, the first bundle 114 is threaded over the proximal (rearward) end by passing the apertures 112 over the wire guide 118. A plastic tube 122 is then positioned over the proximal end of the wire guide 118, and slid towards the distal end of the wire guide 118 so as to push the folds 104 of the first bundle against the nylon tubing 120. This procedure is then repeated by threading subsequent bundles 114 over the wire guide 118 and pushing them against the previously inserted bundles 114 until all of the bundles 114 have been inserted into the gastric lumen. The bundles 114 are then secured together by pushing a small rubber stopper or similar device 124 (see FIG. 15) along the wire guide 118 so as to press against the last bundle 114 to be inserted. The wire guide 118 is then withdrawn so as to leave the cinching member 110 extending through the apertures 112 of all of the bundles 114. The cinching member 110 is then tied or otherwise secured to the stopper 124 so as to form a complete intragastric member 100 as shown in FIG. 15.

To remove the intragastric member 100 from the gastric lumen, the cinching member 110 is typically cut so as to release the folds 104. One end of the strip 102 is then grasped by an endoscopic or similar device and pulled out of the patient.

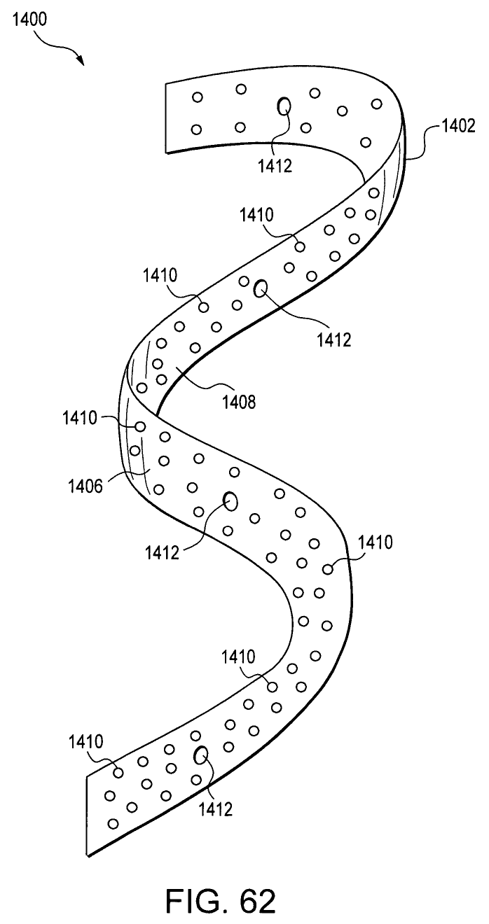

FIG. 62 depicts another embodiment of an intragastric member 1400 of the present invention. In this embodiment, the intragastric member 1400 comprises a single strip of high-density polyethylene 1402 having a plurality of openings 1410 positioned along the length of the strip 1402. The plurality of openings 1410 reduce the overall mass of the intragastric member 1400 and also decreases the total thickness of the intragastric member 1400 after the strip 1402 is bundled in the gastric lumen. As best seen in FIG. 62, the single strip of high-density polyethylene 1402 of the embodiment is formed from a sheet of material having a wall thickness of 7.5 microns and a perimeter of 6 cm. The single strip 1402 comprises a first side 1406 and an opposite side 1408 and is formed from a sheet of material. The intragastric member 1400 further comprises a plurality of apertures 1412 positioned along the center of the strip 1402. The length of the intragastric member 1400 may vary depending on the particular shape and design.

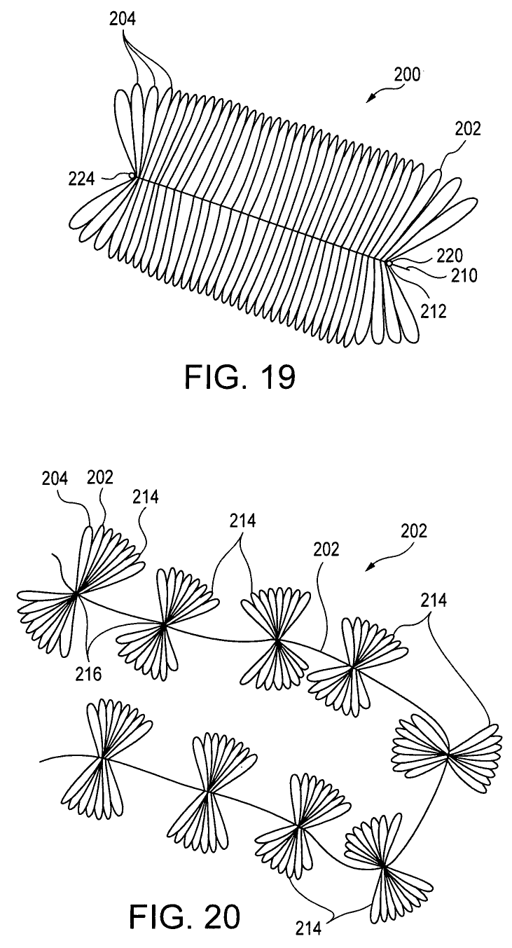

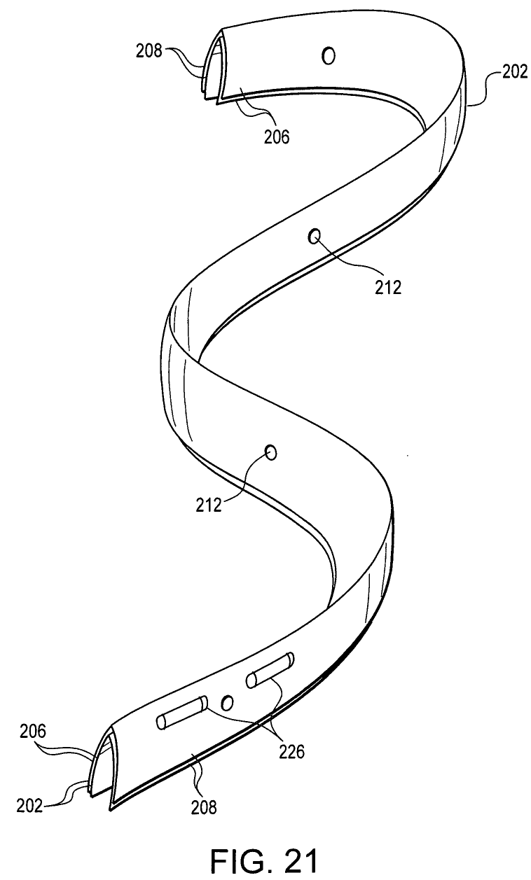

FIG. 19 depicts yet another embodiment of an intragastric member 200 of the present invention. In this embodiment, the intragastric member 200 comprises a double strip of low-density polyethylene 202 that has been folded and bundled to form approximately forty-five (45) loops 204 in the general shape of a butterfly or bow-tie. A double strip of low-density polyethylene 202 is defined as a first strip of low-density polyethylene bundled with a second strip of low-density polyethylene (see FIG. 21). The double strip of low-density polyethylene 202 of this embodiment comprises a pair of strips 202 each having two walls 206, 208, wherein each wall 206, 208 has a width of 15 mm and thickness in the range of 40-50 microns.

In the embodiment of the intragastric member 200 shown in FIG. 19, each loop 204 is 20 cm in length. Accordingly, the intragastric member 200 is formed from a double strip 202 of material having a total length of approximately 18 m (i.e., each strip 202 has a total length of approximately 18 m). A double strip 202 having longer or shorter lengths may also be used depending on the desired size and mass of the intragastric member 200.

The intragastric member 200 is bundled by passing a cinching member 210, such as a nylon thread or similar thread-like structure, through an aperture 212 in each strip 202 at the center of the each loop 204. As best seen in FIG. 21, the apertures 212 are formed in each wall 206, 208 of each strip 202, and are spaced so that loops 204 are formed 20 cm in length when adjacent apertures 212 are pulled together to form the intragastric member 200 shown in FIG. 19. In other words, the apertures 212 are located every 20 cm along the length of the strip 202. In the preferred embodiment shown, apertures 212 have a diameter of approximately 3.5 mm.

The embodiment of the intragastric member 200 shown in FIG. 19 may be too large for delivery or insertion into the gastric lumen while in its bundled, final configuration. Accordingly, the intragastric member 200 is preferably inserted into the gastric lumen is stages. For example, and as shown in FIG. 20, the intragastric member 200 is separated into nine (9) separate bundles 214, each of which comprise approximately five (5) loops 204 of the strip 202. The loops 204 of each separate bundle 214 are grouped or held together by a breakable tie 216, made of cotton thread, or similar device. As will be explained below, grouping the separate bundles 214 in this manner improves the handling of the material and prevents the strips 202 from becoming tangled or contaminated during the insertion thereof.

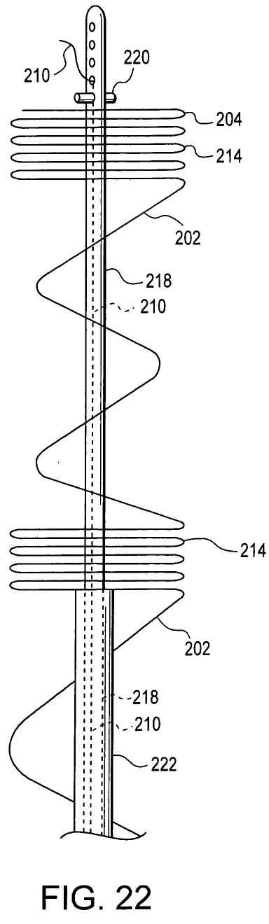

As shown in FIG. 22, the separate bundles 214 of the intragastric member 200 are inserted into the gastric lumen one at a time by using a wire guide 218 such as a Savary-Gilliard™ wire guide, manufactured by Cook Endoscopy, Winston-Salem, N.C. The wire guide 218 comprises a central opening through which the cinching member 210 passes. The end of the cinching member 210 is connected to or tied around a small nylon disc 220 that is sized so as to not pass through the apertures 212 in the strips 202. Prior to the insertion procedure, the nylon disc 220 is placed near the distal (forward or insertion) end of the wire guide 218 so as to prevent the strips 202 of the first bundle 214 from sliding off the end of the wire guide 218.

Once the distal end of the wire guide 218 is positioned in the gastric lumen, the first bundle 214 is threaded over the proximal (rearward) end by passing the apertures 212 over the wire guide 218. A pusher tube 222, which may be plastic, metal or some other suitable material, is then positioned over the proximal end of the wire guide 218, and slid towards the distal end of the wire guide 218 so as to push the folds 204 of the first bundle 214, which remain bundled by tie 216, against the nylon disc 220.

In the preferred embodiment shown, one or more of the apertures 212 in each bundle 214 have an increased diameter that is sufficient to allow one more folds 204 to slide over the outside of the pusher tube 222. This permits the portion of the strips 202 connected between adjacent bundles 214 to be guided (extended) along the wire guide 218 without interfering with the deployment of each bundle 214. In the preferred embodiment shown, those apertures 212 having an increased diameter are approximately 9-10 mm in diameter.

This procedure is then repeated by threading subsequent bundles 214 over the wire guide 218 and pushing them against the previously inserted bundles 214 until all of the bundles 214 have been inserted into the gastric lumen. The bundles 214 are then secured together by pushing a small rubber stopper or similar device 224 (see FIG. 19) along the wire guide 218 so as to press against the last bundle 214 to be inserted. The wire guide 218 is then withdrawn so as to leave the cinching member 210 extending through the apertures 212 of all of the bundles 214. The cinching member 210 is then tied or otherwise secured to the stopper 224 so as to form a complete intragastric member 200 as shown in FIG. 19.

To remove the intragastric member 200 from the gastric lumen, the cinching member 210 is typically cut so as to allow the intragastric member 200 to separate in separate bundles (see FIG. 20). The separate bundles 214, which remain connected to each other by strips 202, can then be removed one at a time. In the event that the removal of the intragastric member 200 in separate bundles 214 becomes difficult or problematic, then breakable ties 216 may be severed to release the folds 204 of one or more of the bundles 216.

As best seen in FIG. 21, visual markers 226, such as colored tubing, are sutured to the side of the strips 202 of the first or last fold 204 on either side of the aperture 212. These markers 226 assist the physician in locating the cinching member 210, which may be difficult to identify after the device has resided within the gastric lumen for an extended period of time. Once the cinching member 210 is cut, one end of the pair of strips 202, or one of the bundles 216, is then grasped by an endoscopic or similar device and pulled out of the patient.

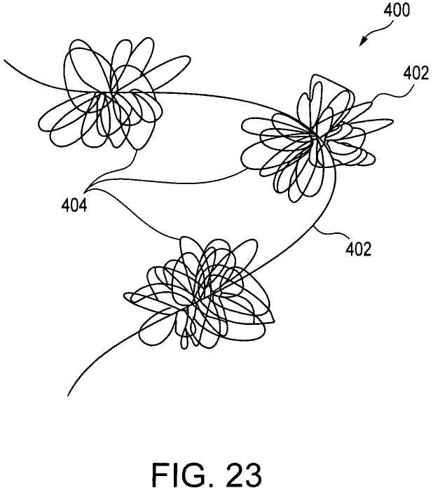

FIG. 23 depicts yet another embodiment of an intragastric member 400 of the present invention. In this embodiment, the intragastric member 400 comprises nylon thread 402 or similar thread-like structure that has been tied into a series of nylon balls 404. The nylon balls 404 are inserted into the gastric lumen separately and then connected together to form a single, larger mass of nylon thread (not shown).

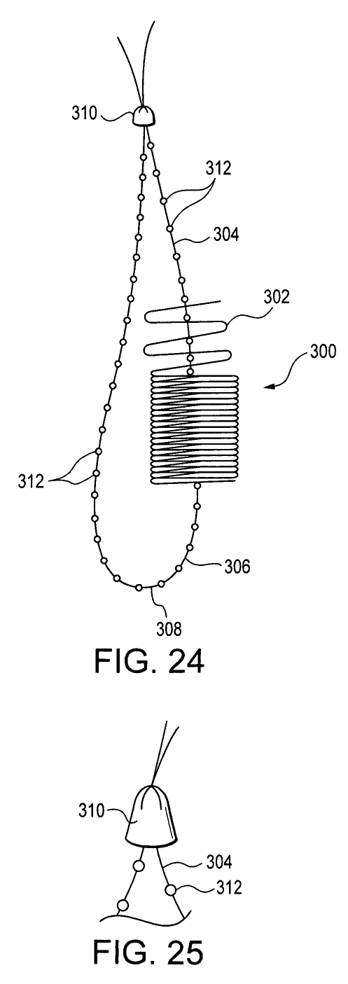

The above-described embodiments, particularly the embodiments of FIGS. 15 and 19, can be deployed using alternative procedures. For example, and as shown in FIGS. 24 and 25, the intragastric member 300 could be deployed by extending the strip 302 along a cinching member 304, such as nylon thread or similar thread-like structure, that has been formed into a loop 306. Once the end 308 of the loop 306 has been inserted into the gastric lumen, then a locking device 310, such as plastic cone (shown in detail in FIG. 25), is pushed over both strands of the cinching member 304 so as to close the loop 306. As the loop 306 is closed, the strip 302 is compressed so as to form an intragastric member 300 having a configuration similar to that shown in FIGS. 15 and 19. Knots 312 are included along the cinching member 304 to provide a ratcheting action with the locking device 310. After the intragastric member 300 has been deployed inside the gastric lumen, then the portion of the cinching member 304 extending beyond the locking device 310 can be severed with an endoscopic scissors and removed.

Alternatively, the strip 302 can be compressed by sliding a tube (not shown) along one or both halves of the loop 306. In addition, the intragastric member 300 can be inserted in bundles (see FIGS. 16 and 20), as opposed to the insertion of a single strip 302 of material (as described above).

An anchor stent (not shown) could be utilized to temporarily secure the end of the cinching member 304 (or the end 308 of the loop 306) inside the gastric lumen during the insertion procedure. For example, an anchor stent enclosing a portion of the cinching member 304 would be inserted into the pylorus and lodged therein. One end of the cinching member 304 (or loop 306) enclosed within the anchor stent is then removed therefrom and pulled out of the subject. The other end of the cinching member 304 (or loop 306) remains attached to the anchor stent. The intragastric device 300 can then be inserted into the gastric lumen by pushing or sliding the strip 302 (or bundles) down the cinching member 304 (or loop 306), the end of which remains secured within the gastric lumen by the anchor stent. Once the insertion procedure is removed, then the anchor stent and any excess cinching member 304 are removed.

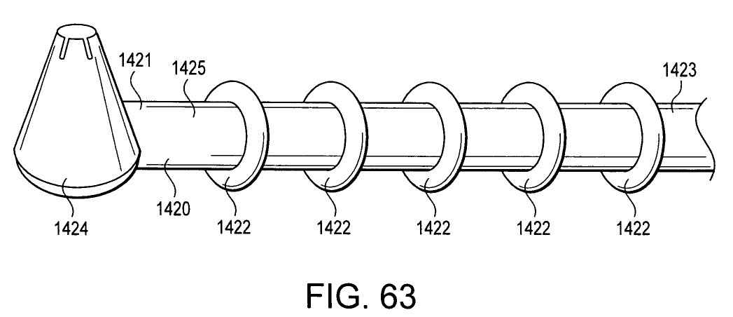

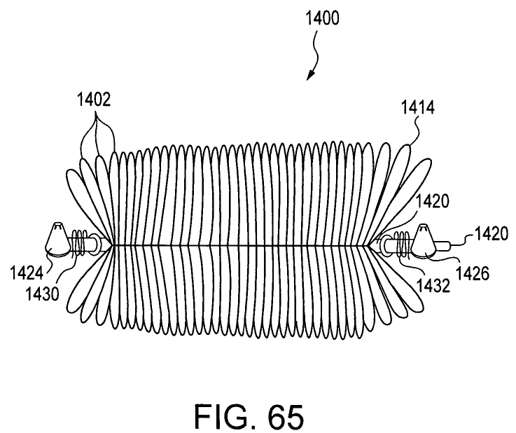

FIGS. 63-65 depict yet another embodiment of the present invention. In this embodiment, the intragastric member 1400 (see FIG. 65) utilizes an elongate member 1420 to deliver and secure the intragastric member 1400 into the gastric lumen of the patient. The intragastric member 1400 is bundled by passing the elongate member 1420 through the aperture 1412 in the strip 1402 (FIG. 65) upon delivery into the gastric lumen of the patient. The elongate member 1420 can be attached to a distal end of a wire guide to facilitate delivery, as shown in FIG. 18. The intragastric member 1400 is bundled onto the elongate member 1420 to deliver the intragastric member 1400 into the gastric lumen in stages and allow the loops to be pulled together to form a secured intragastric member 1400 upon delivery to the lumen of the patient.

In this embodiment, the elongate member 1420 comprises a plurality of ribs 1422 dispensed along the length of the elongate member 1420. The ribs 1422 are spaced along the elongate member 1420 to engage the intragastric member 1400 after insertion into apertures 1412 of the intragastric member 1400. The elongate member 1420 further comprises a distal stopper 1424, such as a pawl, engaged to a distal end 1421 of the elongate member 1420 and a proximal stopper 1426, such as a second pawl, attached to a proximal end 1423 of the elongate member 1420. The ribs 1422 provide a ratcheting action upon engagement with a lumen 1419 of the proximal stopper 1426, similar to a pawl mechanism. The ribs 1422 also allow the elongate member 1420 to be pulled only in a proximal direction upon engagement with the proximal stopper 1426.

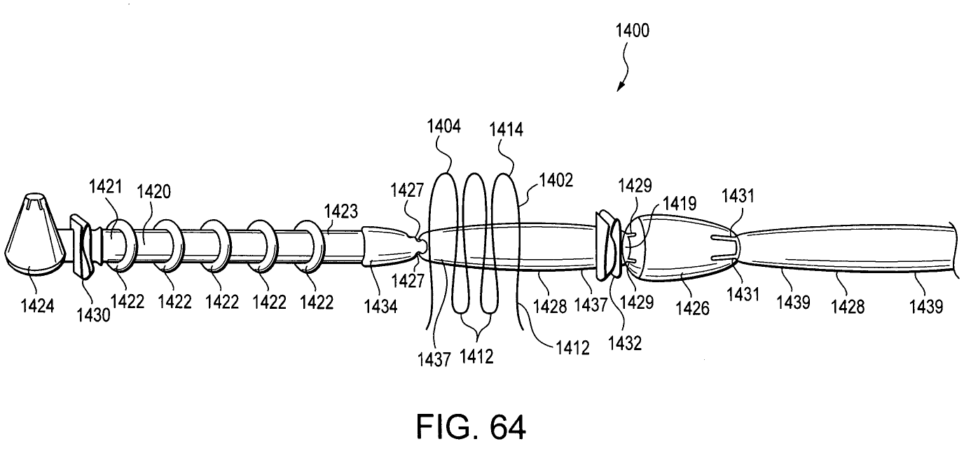

As best shown in FIG. 64, the proximal stopper 1426 is engaged to the elongate member 1420 utilizing a cinching member 1428, such as a nylon thread or similar thread-like structure. The cinching member 1428 comprises a proximal end 1439 and a distal end 1437. The distal end 1437 of the cinching member 1428 is looped through a lumen 1427 located on a proximal end of a connector 1434 and passes through a lumen 1429 located on a distal end of the proximal stopper 1426. The proximal end 1439 of the cinching member 1428 passes through a lumen 1431 located on the proximal end of the proximal stopper 1426 to facilitate delivery of the intragastric member 1400. The proximal stopper 1426 slides along the cinching member 1428 upon delivery into the gastric lumen. The stoppers 1424, 1426 are utilized to compress the strip 1402 so as to form the intragastric member 1400 having a configuration similar to that shown in FIG. 65.

FIG. 65 depicts an embodiment of the intragastric member 1400 in a bundled, final configuration after delivery or insertion into the gastric lumen. Accordingly, the intragastric member 1400 is preferably inserted into the gastric lumen in stages. For example, and as shown in FIG. 64, the intragastric member 1400 is separated into a plurality of separate bundles 1414 to improve the handling of the material and prevent the strip 1402 from becoming tangled or contaminated. The separate bundles 1414 of the intragastric member 1400 are delivered to the elongate member 1420 one at a time after being inserted into the gastric lumen. Before delivery, a second distal stopper 1430, such as a drag, is engaged to the distal end 1421 of the elongate member 1420 between the distal stopper 1424 and the first rib 1422. A second proximal stopper 1432, such as a second drag, is connected to the proximal end 1439 of the cinching member 1428 between the connector 1434 and proximal stopper 1426 after delivery of all the bundles 1414 to the lumen. Both the second distal stopper 1430 and the second proximal stopper 1432 are sized and configured so as to not pass over the stoppers 1424, 1426 or through the apertures 1412 in the strip 1402.

Once a distal end 1421 of the elongate member 1420 is positioned in the gastric lumen, the first bundle 1414 is threaded over the proximal (rearward) end by passing the apertures 1412 over the elongate member 1420. A pusher (not shown) is then positioned over the proximal end of the elongate member 1420, and slid towards the distal end 1421 of the elongate member 1420 so as to push the folds 1404 of the first bundle against the second distal stopper 1430 and the distal stopper 1424. This procedure is then repeated by threading subsequent bundles 1414 over the elongate member 1420 and pushing them against the previously inserted bundles 1414 until all of the bundles 1414 have been inserted into the gastric lumen (see FIG. 65). The bundles 1414 are then secured together by pushing the proximal stopper 1426 distally to fasten the bundles 1414. The proximal stopper 1426 is passed along the ribs 1422 of the elongate member 1420 providing tactile confirmation that the proximal stopper 1426 is securely fastened. The cinching member 1428 is subsequently removed and the delivery device, such as the wire guide, is then withdrawn so as to leave the intragastric member 1400 secured along the elongate member 1420. To remove the intragastric member 1420 from the gastric lumen, the elongate member 1420 is typically cut so as to release the folds 1404. One end of the strip 1402 is then grasped by an endoscopic or similar device and pulled out of the patient.

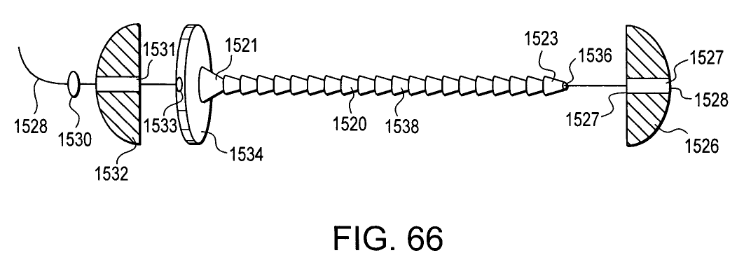

FIGS. 66-68 depict another embodiment of the present invention. In this embodiment, an elongate member 1520 is used to deliver the intragastric member 1400 into the gastric lumen of the patient. In this embodiment, the elongate member 1520 includes a plurality of ribs 1538 engaged between a proximal end 1523 and a distal end 1521 of the elongate member 1520. The elongate member 1520 further comprises a lumen 1536 configured to receive a cinching member 1528, such as a nylon thread or similar thread-like structure, to facilitate delivery of the intragastric member 1400.

The elongate member 1520 further comprises a distal stopper 1532, such as a button, engaged to the distal end 1521 of the elongate member 1520 and a proximal stopper 1526, such as a second button, engaged to a proximal end 1523 of the elongate member 1520. The proximal stopper 1526 includes a lumen 1527 configured to receive the cinching member 1528 that passes through the lumen 1536 of the elongate member 1520. The distal end 1521 of the elongate member 1520 also includes a locking member 1534 that engages a portion of the intragastric member 1400 after complete delivery of the intragastric member 1400. The distal stopper 1532 and the locking member 1534 engage the strip 1402 of the intragastric member 1400 and trap the intragastric member 1400 between the distal stopper 1532 and the locking member 1534 to secure the intragastric member 1400 upon delivery into the gastric lumen. Both the distal stopper 1532 and the locking member 1534 include a lumen 1531, 1533 configured to receive the cinching member 1528 that passes through the lumen 1536 of the elongate member 1520. In addition, a secondary distal stopper 1530 is engaged along the cinching member 1528 adjacent the distal end of the distal stopper 1532 to further secure the intragastric member 1400 upon delivery.

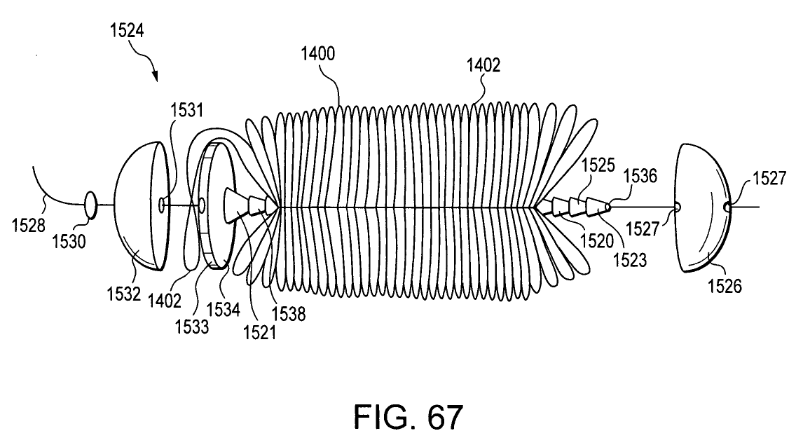

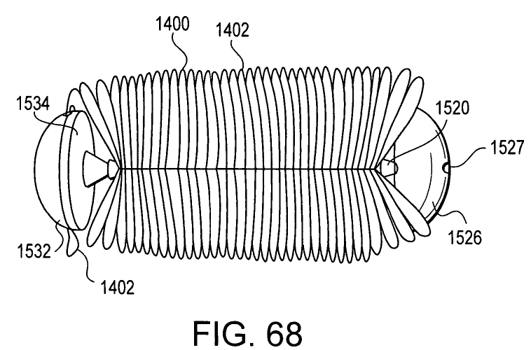

FIG. 67 depicts the elongate member 1520 after delivery of the intragastric member 1400 into the gastric lumen. In this embodiment, the intragastric member 1400 is deployed by extending the strip 1402 along the elongate member 1520 by pushing the intragastric member 1400 over the elongate member 1520, so as to secure the intragastric member 1400 along the distal stopper 1532 and the elongate member 1520. The elongate member 1520 is then secured and closed by engaging the ribs 1538 of the elongate member 1520 to the lumen 1527 of the proximal stopper 1526. Ribs 1538 are included along the elongate member 1520 to provide a ratcheting action upon engagement with the proximal stopper 1526. The ribs allow the elongate member 1520 to pass only in a forward direction upon engagement and prevent the elongate member 1520 from moving in an opposite direction after engagement with the lumen 1527 of the proximal stopper 1526. As the elongate member 1520 is closed, the strip 1402 is compressed so as to form the intragastric member 1400 having a configuration similar to that shown in FIGS. 67 and 68. After the intragastric member 1400 has been secured inside the gastric lumen, then the portion of the elongate member 1520 extending beyond the proximal stopper 1526 can be severed with an endoscopic scissors and removed. In addition, the intragastric member 1400 can be inserted in bundles, as opposed to the insertion of a single strip 1402 of material (as described above).

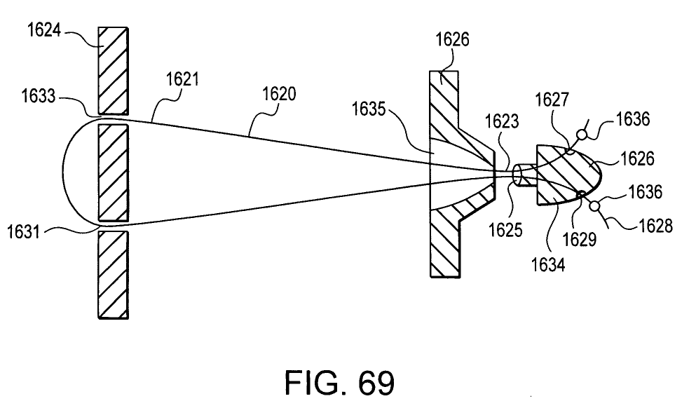

FIG. 69 depicts yet another embodiment of the present invention. In this embodiment, a cinching member 1620 is used to deliver the intragastric member 1400 into the gastric lumen of the patient. The cinching member 1620 includes a distal stopper 1624, such as a button, and a proximal stopper 1626, such as a second button, wherein the distal stopper 1624 is engaged to a distal end 1621 of the cinching member 1620 and the proximal stopper 1626 is engaged to a proximal end 1623 of the cinching member 1620. The cinching member 1620 is engaged to the distal stopper 1624 by looping the cinching member 1620 through a first lumen 1631 and a second lumen 1633 of the distal stopper 1624.

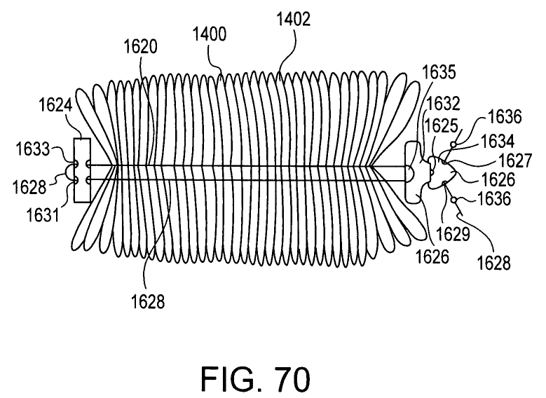

As shown in FIG. 69, a secondary proximal stopper 1634 is engaged along the cinching member 1620 adjacent the proximal end of the proximal stopper 1626 to further secure the intragastric member 1400 upon delivery. The proximal stopper 1626 comprises a lumen 1635 configured to receive the cinching member 1620 as it passes from the distal stopper 1624. The lumen 1635 further operates as a point of connection to interlock the secondary proximal stopper 1634 to the proximal stopper 1626 after the stoppers 1634, 1626 are fastened after complete delivery of the intragastric member 1400 (FIG. 70). The secondary proximal stopper 1634 further comprises a first lumen 1625, a second lumen 1627 and a third lumen 1629, wherein the first lumen 1625 receives the cinching member 1620 as it is passes from the lumen 1635 of the proximal stopper 1626, and wherein the second lumen 1627 and the third lumen 1629 receive the cinching member 1620 as it exits the first lumen 1625 of the second portion 1634.

FIG. 70 depicts the cinching member 1620 after delivery of the intragastric member 1400 in a bundled, final configuration. In this embodiment, the intragastric member 1400 is deployed by extending the strip 1402 along the cinching member 1620 after the intragastric member 1400 has been delivered into the gastric lumen and the intragastric member 1400 is pushed over the cinching member 1620, so as to secure the intragastric member 1400 along the cinching member 1620. The proximal stopper 1626 and second proximal stopper 1634 are closed by engaging a distal end of the second proximal stopper 1634 to the lumen 1635 of the proximal stopper 1632 and passing the proximal end 1623 of the cinching member 1620 through the second lumen 1627 and third lumen 1625 of the second proximal stopper 1634. After the cinching member 1620 is closed, the strip 1402 is compressed so as to form the intragastric member 1400 having a bundled, final configuration. The ends of the cinching member 1620 can be tied in knots 1636 to further secure the intragastric member 1400 in the gastric lumen. After the intragastric member 1400 has been secured inside the gastric lumen, then the portion of the cinching member 1620 extending beyond the second proximal stopper 1634 can be severed with an endoscopic scissors and removed. In addition, the intragastric member 1400 can be inserted in bundles, as opposed to the insertion of a single strip 1402 of material (as described above).

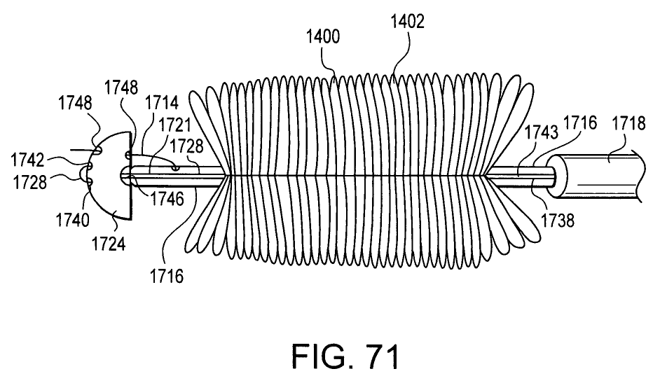

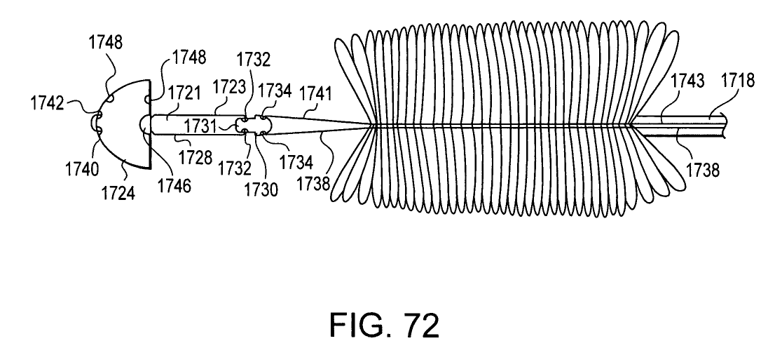

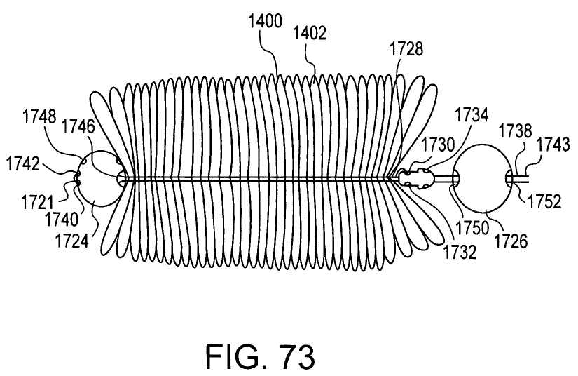

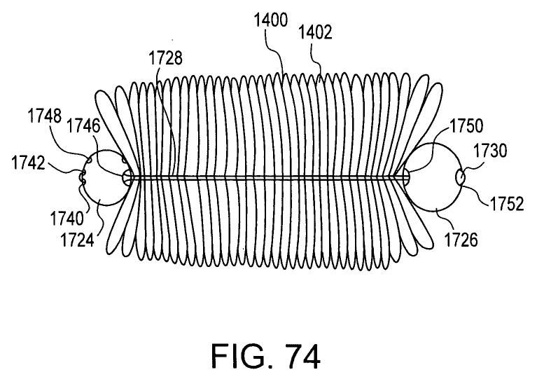

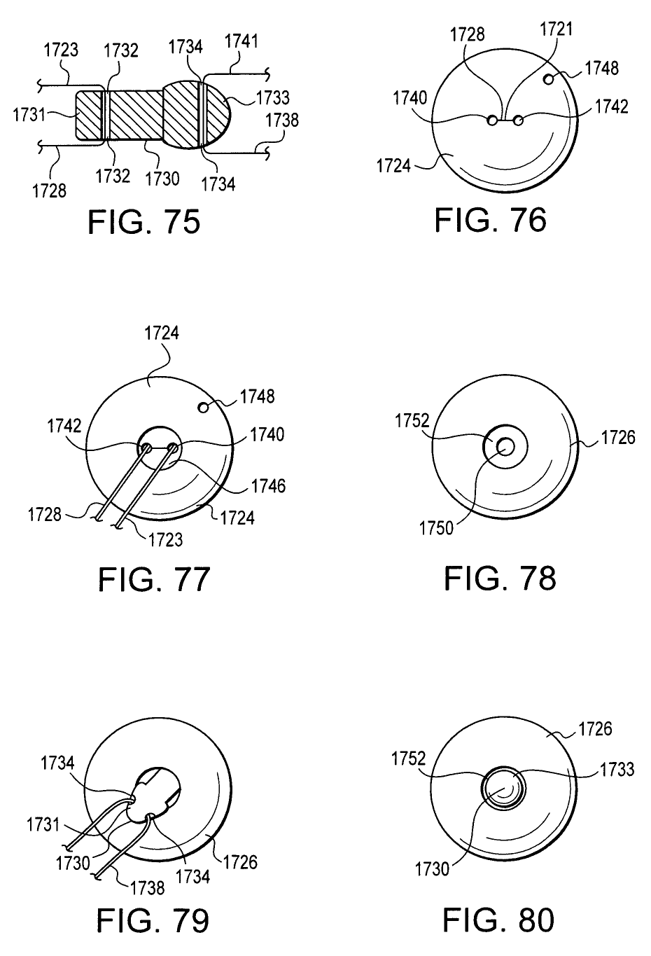

FIGS. 71-80 depict yet another embodiment of the intragastric member 1400 of the present invention. In this embodiment, the intragastric member 1400 utilizes a first cinching member 1728 and a second cinching member 1738, such as a nylon thread or similar thread-like structure, to deliver the intragastric member 1400 into the gastric lumen of the patient. Similar to the above embodiments, the intragastric member 1400 may be loaded and delivered to the first cinching member 1728 using a wire guide 1714, an inner delivery tube 1716 and an outer delivery tube 1718, or other similar delivery devices (FIG. 71). As shown in FIG. 72, the first cinching member 1728 comprises a proximal end 1723 and a distal end 1721 wherein the intragastric member 1400 is threaded between the proximal end 1723 and the distal end 1721 of the first cinching member 1728. A distal stopper 1724, such as a button, is disposed on the distal end 1721 of the first cinching member 1728 for securing the intragastric member 1400 along the first cinching member 1728 (FIG. 73). A proximal stopper 1726, such as a second button, is disposed on the proximal end 1723 of the first cinching member 1728 for locking the intragastric member 1400 along the first cinching member 1728 (FIG. 74). The first cinching member 1728 is further engaged to a distal end 1731 of a second proximal stopper 1730, such as a bead, and the second cinching member 1738 is engaged to a proximal end 1733 of the second proximal stopper 1730 (FIG. 75).

As illustrated in FIGS. 76-77, the distal stopper 1724 is engaged to the first cinching member 1728 by looping the first cinching member 1728 through a first lumen 1732 of the second proximal stopper 1730. The distal stopper 1724 comprises a first lumen 1746, a second lumen 1740 and a third lumen 1742, wherein the first cinching member 1728 enters the first lumen 1746 and loops through the second lumen 1740 and third lumen 1742 before exiting the first lumen 1746 to form a complete loop with the first lumen 1732 of the second proximal stopper 1730. The first lumen 1732 also provides an opening to secure the distal stopper 1724 to the inner delivery tube 1716 during delivery. In addition, the distal stopper 1724 comprises a fourth lumen 1748 for securing the wire guide 1714 during delivery.

As illustrated in FIGS. 78-80, the proximal stopper 1726 is engaged to the second cinching member 1738 by looping the second cinching member 1738 through a second lumen 1734 of the second proximal stopper 1730. The proximal stopper 1726 comprises a first lumen 1750 and a second lumen 1752 for receiving the second proximal stopper 1730. The first lumen 1750 comprises a first diameter and the second lumen 1752 comprises a second diameter wherein the first diameter is smaller than the second diameter. The first diameter is configured to allow the second proximal stopper 1730 to only pass in a proximal direction through the first lumen 1750 of the proximal stopper 1726 (FIG. 78), and prevents the second proximal stopper 1730 from passing distally back through the first lumen 1750 (FIG. 79). Therefore, once the second proximal stopper 1730 passes through the first lumen 1750 it is secured in the first lumen 1750 (FIGS. 78-80).

FIG. 74 depicts the first cinching member 1728 after delivery of the intragastric member 1400 in a bundled, final configuration. In this embodiment, the intragastric member 1400 is deployed by extending the strip 1402 along the first cinching member 1728 after the intragastric member 1400 has been delivered into the gastric lumen and the intragastric member 1400 is pushed over the first cinching member 1728, so as to secure the intragastric member 1400 along the first cinching member 1728. The distal stopper 1724 and the proximal stopper 1726 are closed by pulling the second cinching member 1738 proximally until the second proximal stopper 1730 fully engages the first lumen 1750 of the proximal stopper 1726. After the first cinching member 1728 is closed, the strip 1402 is compressed so as to form the intragastric member 1400 having a bundled, final configuration. The second cinching member 1738 is then pulled until it is removed from the intragastric member 1400.

Experimental testing of the present invention has been conducted on mammals. In particular, an embodiment of an intragastric member similar to the embodiment shown in FIGS. 19-21 and FIGS. 64-74 was inserted into the gastric lumens of a group of ten (10) pigs for a period of 49 days. No deaths or major complications were observed in any of the test subjects. The initial weight for the test subjects was measured to be in the range of 25.0 to 31.2 kg, with an average weight of 27.8 kg. At the end of the 49 day testing period, the weight of the test subjects was measured to be in the range of 29.5 to 39.0 kg, with an average weight of 34.5 kg. The anticipated weight for the test subjects at the end of the testing period, in view of the normal and expected growth for these animals, was 57 kg. Accordingly, the test subjects gained an average weight that was significantly less than the weight gain observed in similar animals without the intragastric member.

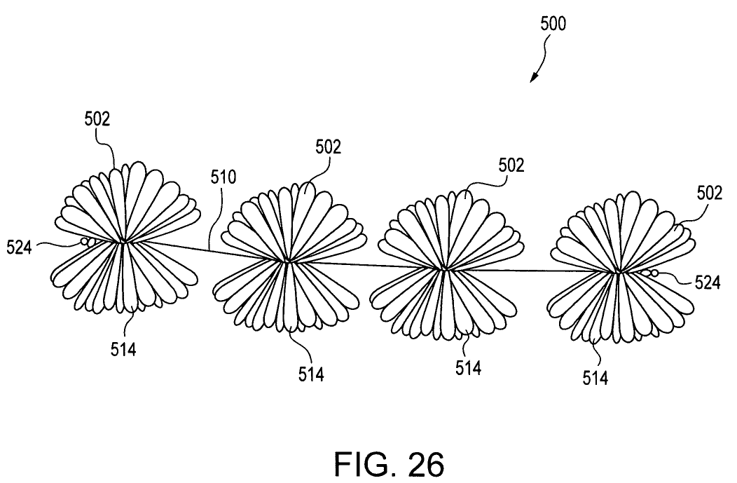

FIG. 26 depicts another embodiment of an intragastric member 500 of the present invention. In this embodiment, the intragastric member 500 comprises a plurality of strips of high-density polyethylene 502 that have been folded and bundled in the general shape of a butterfly or a bow-tie. As best seen in FIG. 34, each strip of high-density polyethylene 502 of the embodiment is formed from a tube of material having a wall thickness of 40-50 microns and a perimeter of 3 cm. The material is then stacked and folded to form a plurality of strips 502 having two walls 506, 508, wherein each wall 506, 508 has a width of 1.5 cm. Of course, the strip 502 could comprise a different number of walls 506, 508, have a different width and thickness, or be formed from a tube of material.

In the embodiment shown in FIG. 26, the intragastric member 500 is formed from approximately five individually stacked strips 502. The intragastric member 500 is bundled by passing a cinching member 510, such as a nylon thread or similar thread-like structure, through an aperture 512 in the strip 502 at the center of the each bundle 514. As best seen in FIG. 34, the apertures 512 are formed in each wall 506, 508 of the strip 502, and are spaced so that bundles 514 are formed 20 cm in length when adjacent apertures 512 are pulled together to form the intragastric member 500 shown in FIG. 26. In other words, the apertures 512 are located every 20 cm along the length of the strip 502. Alternate embodiments of an intragastric member may include varying numbers of strips of high-density polyethylene having longer or shorter lengths depending on the desired size and mass of the intragastric member.

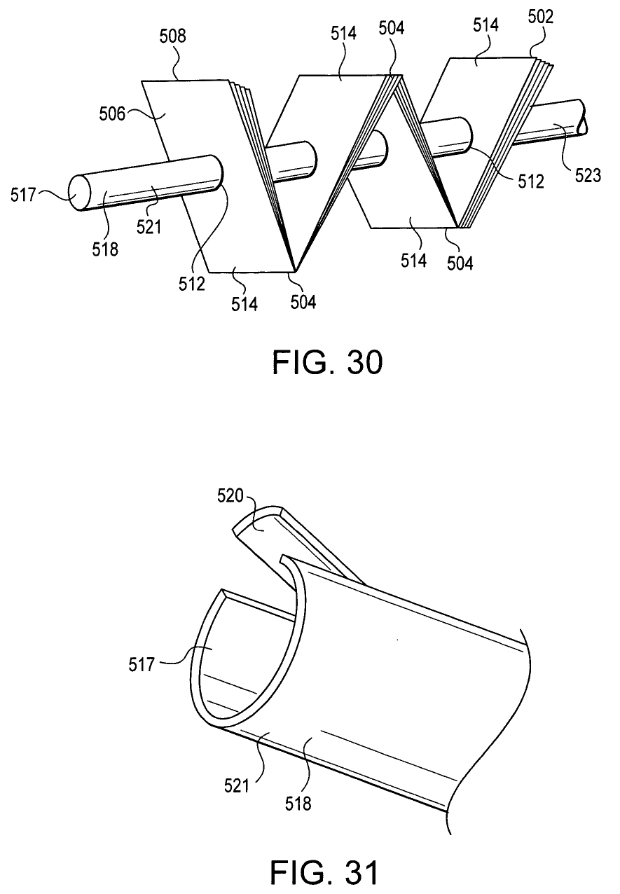

The embodiment of the intragastric member 500 shown in FIG. 26 may be too large for delivery or insertion into the gastric lumen while in its bundled, final configuration. Accordingly, the intragastric member 500 is preferably inserted into the gastric lumen in stages. For example, and as shown in FIG. 30, the intragastric member 500 is separated into four (4) separate bundles 514, each of which comprise approximately five (5) layers of the high-density polyethylene strips 502. The stacked layers of strips 502 provide the bundle 514 with an increased volume, thereby providing a thicker intragastric member 500.

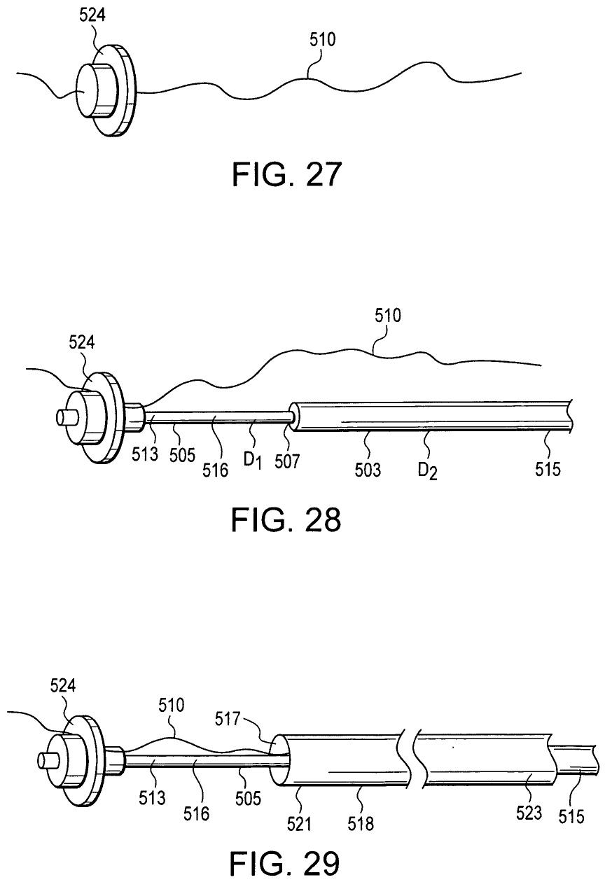

As shown in FIGS. 27-33, the separate bundles 514 of the intragastric member 500 are inserted into the gastric lumen one at a time by loading the intragastric member 500 over an outer delivery tube 518 and delivering through the esophagus via an overtube into the gastric lumen where the strips 502 are gathered in a butterfly or bow-tie formation. The intragastric member 500 is loaded onto the outer delivery tube 518 by threading the outer delivery tube 518 through the apertures 512 of each strip 502 (see FIG. 30). The outer delivery tube 518 comprises a proximal end 523, a distal end 521, and a main opening 517 through which an inner delivery tube 516 passes (see FIG. 29). The inner delivery tube 516 comprises a proximal end 515 and a distal end 513, wherein the distal end 513 is engaged with at least one stopper 524 (see FIG. 28). The stopper 524 is further engaged with the cinching member 510 which passes through the main lumen 517 of the outer delivery tube 518. Delivery of the intragastric member 500 begins by inserting the distal end 513 of the inner delivery tube 516 in the gastric lumen of the patient while the proximal end 515 will remain external to the patient.

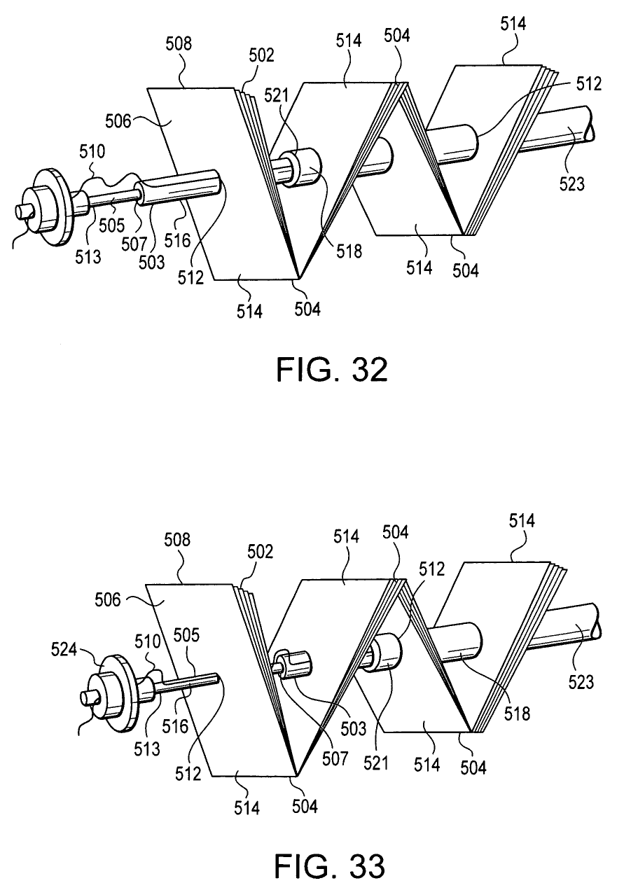

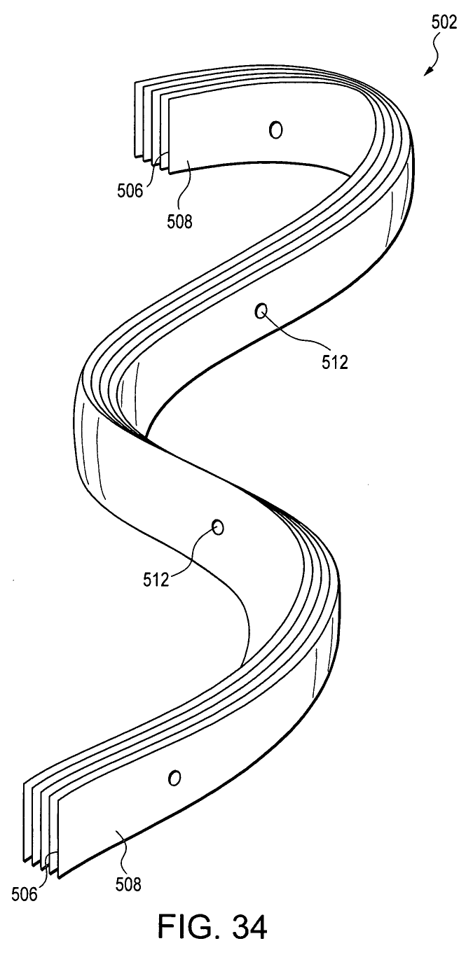

Once the distal end 513 of the inner delivery tube 516 is positioned in the gastric lumen, the first bundle 514 is threaded over the proximal (rearward) end 523 of the outer delivery tube 518 by passing the apertures 512 over the outer delivery tube 518. Each bundle 514 is manually pushed from the proximal end 523 of the outer delivery tube 518, and slid towards the distal end 513 of the inner delivery tube 516 so as to push the folds 504 of the first bundle 514 past the distal end 521 of the outer delivery tube 518 (see FIGS. 32-33). The outer delivery tube 518 comprises a delivery tip 520 that facilitates the one way delivery of each bundle 514 onto the inner delivery tube 516 (see FIG. 31).

The inner delivery tube 516 further comprises a proximal section 503, a distal section 505, and a transitional section 507, wherein the diameter D1 of the distal section 505 may be smaller than the diameter D2 of the proximal section 503 (see FIG. 28). As shown in FIGS. 32-33, the diameter of the proximal section 503 of the inner delivery tube 516 is sized and configured to align with the apertures 512 of the strips 502 after each bundle 514 transitions from the outer delivery tube 518 onto the inner delivery tube 516 during delivery. The plurality of stacked strips 502 further allow the apertures 512 to remain frictionally engaged with the proximal section 503 of the inner delivery tube 516 during delivery. As the outer delivery tube 518 is withdrawn, the subsequent bundle 514 is pushed past the transitional section 507 of the inner delivery tube 516 onto the distal section 505. Each bundle 514 is generally retained in the distal section 505 of the inner delivery tube 516, wherein the length of the distal section 505 is approximately equivalent to the length of the bundle 514. The varying diameter of the inner delivery tube 516 combined with the stacked strips 502 allow the bundles 514 to be easily delivered. A skilled artisan would appreciate that other designs comprising an inner delivery tube of different diameters could be utilized.

This procedure is then repeated by threading subsequent bundles 514 over the outer delivery tube 518 and pushing them against the previously inserted bundles 514 until all of the bundles 514 have been inserted into the gastric lumen (see FIG. 33). The bundles 514 are then secured together by pushing a proximal stopper or similar device (see FIG. 26) along the outer delivery tube 518 so as to press against the last bundle 514 to be inserted. The outer delivery tube 518 is then withdrawn so as to leave the cinching member 510 extending through the apertures 512 of all of the bundles 514. The cinching member 510 is then tied or otherwise secured to the proximal stopper so as to form a complete intragastric member 500 as shown in FIG. 26. For example, in an alternate embodiment, the stopper can be secured by crimping together a first end and second end of the cinching member 510 with a metal band, such as a ferrule.

To remove the intragastric member 500 from the gastric lumen, the cinching member 510 is typically cut so as to release the folds 504. One end of the strip 502 is then grasped by an endoscopic or similar device and pulled out of the patient.

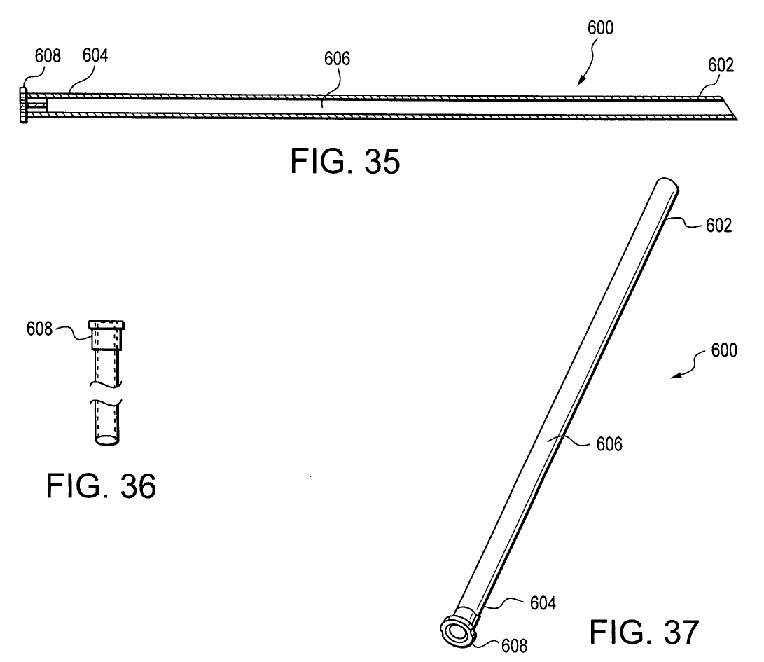

FIG. 35 depicts an overtube 600 that is used to deliver an intragastric member 500 to the gastric lumen of the patient. The overtube 600 is used in combination with an endoscope to establish a passageway to a target delivery site in the stomach. Once the overtube 600 is positioned in the gastric lumen of the patient, the intragastric member 500 is passed through the overtube 600, and is used to deliver the intragastric member 500 to the gastric lumen. Once the desired delivery in the gastric lumen is complete, the overtube 600 is removed.

The overtube 600 comprises a proximal end 604, a distal end 602, and a main lumen 606. Any arrangement of the main lumen 606 is contemplated. FIGS. 35 and 37 illustrate an embodiment of the overtube 600 wherein the overtube 600 is flexible and includes a single-piece construction. Alternatively, several tubes may be bonded together to form the flexible overtube 600 (see FIG. 36). The overtube 600 can be made from any suitable material known in the art including, but not limited to, polyethylene ether ketone (PEEK), polytetrafluorethylene (PTFE), polyamide, polyurethane, polyethylene and nylon, including multi-layer or single layer structures and may also include reinforcement wires, braid wires, coils and or filaments. In the embodiment of the overtube 600 shown in FIG. 42, the overtube 600 comprises a reinforcement member 612, such as metal coil 614, molded into the main lumen 606 of the overtube 600. The reinforcement member 612 provides structural reinforcement to the overtube 600, thereby decreasing the minimum radius that the overtube 600 can withstand without kinking or otherwise reducing the inside diameter of the overtube 600.

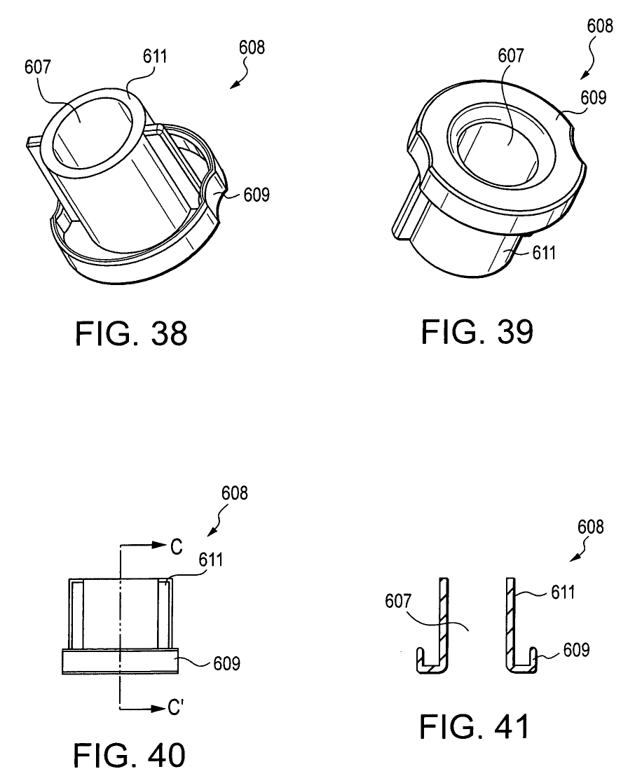

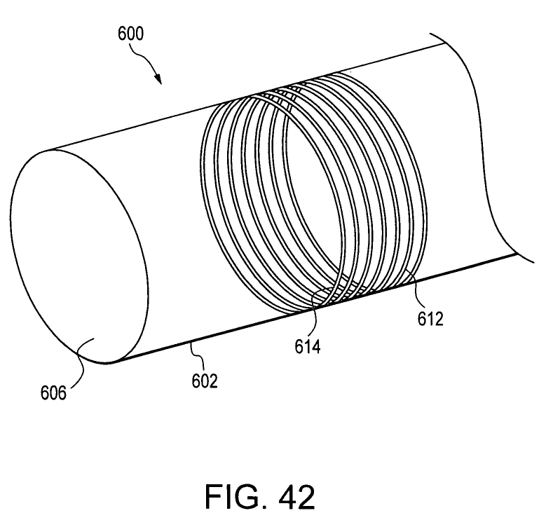

Optionally, the overtube 600 further comprises a valve 608 over its proximal end 604, as illustrated in FIGS. 35-41. The valve 608 forms a seal between the flexible overtube 600 and any secondary device, such as an endoscope or the like, that is advanced therethrough to prevent the loss of any fluid that is introduced through the endoscope or other device. In one embodiment, the valve 608 is removable. The valve 608 has a proximal end 609 and a distal end 611 (see FIGS. 38-39), wherein the distal end 611 is engaged with the proximal end 604 of the overtube 600 (see FIG. 35). The valve 608 further includes a lumen 607 which provides an opening for passing a secondary device (see FIG. 38), such as an endoscope or intragastric member 500. The valve 608 also provides a connection point for engaging any suitable secondary device used in connection with the overtube 600. One of ordinary skill in the art would know how to assemble valve 608 over the proximal end 604 of overtube 600.

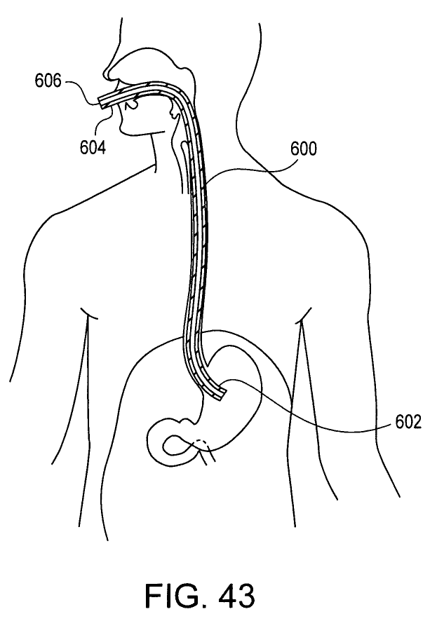

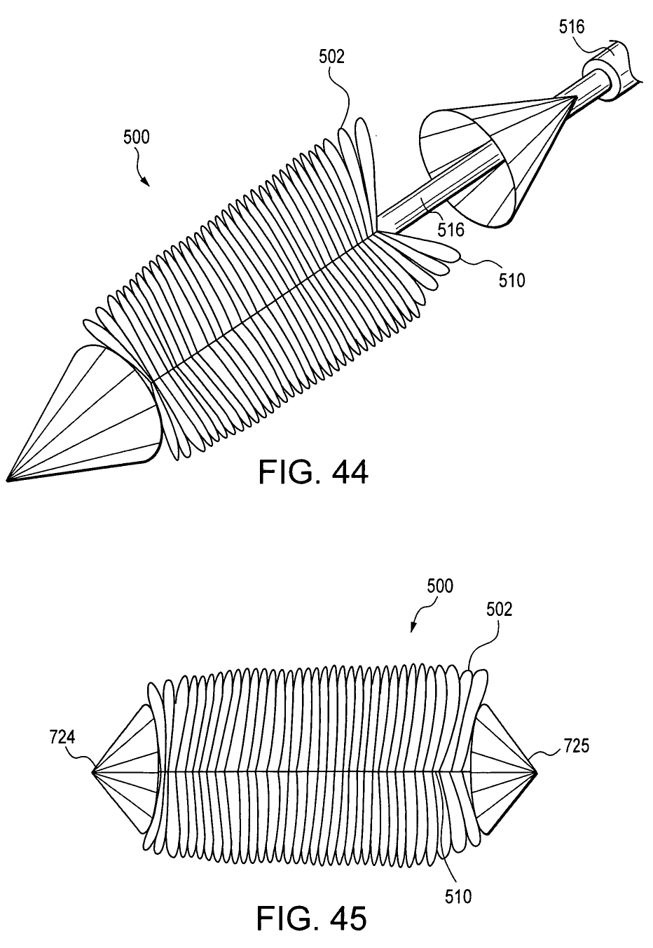

The main lumen 606 is configured to receive and pass an intragastric member 500, or suitable secondary device, such as an endoscope (see FIG. 43). The main lumen 606 ranges in size depending on the size of the intragastric member 500 deployed. The size of the overtube 600 and corresponding intragastric member 500 are provided for illustrative purposes only and are not intended to be construed as a limitation of the present invention. As one of ordinary skill in the art would appreciate, since the intragastric member 500 and the endoscope and are advanced through the main lumen 606, the size of the main lumen 606 is related to the size of either the intragastric member 500 or the endoscope, which ever is larger. One of ordinary skill in the art would also appreciate that the size of the intragastric member 500 is related to the length, width, and number of polyethylene strips comprising the intragastric member 500. Thus, a flexible overtube 600 may have smaller or larger dimensions depending on the size of the intragastric member 500, endoscope or other secondary device used in conjunction with the overtube 600 and therefore any overtube 600 of varying dimensions is contemplated as being within the scope of the claims of the present invention.

FIGS. 44-45 depict another embodiment of the present invention wherein the intragastric member 500 comprises a distal stopper 724 engaged with a distal end of the intragastric member 500 and a proximal stopper 725 engaged with a proximal end of the intragastric member 500 to further secure the intragastric member 500 in its intended formation. In this embodiment, the distal stopper 724 and the proximal stopper 725 comprise a pair of preformed expandable stoppers having an umbrella-like shape.

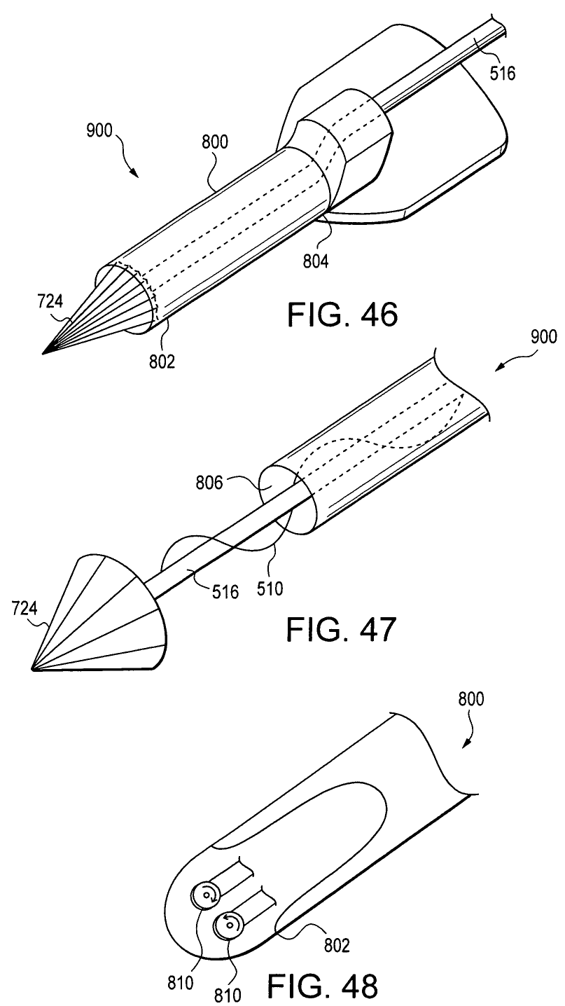

During delivery, the distal stopper 724 is engaged with an end of the inner delivery tube 516 and cinching member 510 while in an unexpanded first position (see FIG. 46). The distal stopper 724 passes through the main lumen of an overtube during delivery and upon delivery of the intragastric member 500 into the gastric lumen, the distal stopper 724 expands to a second position wherein the diameter of the distal stopper 724 is suitable to retain the corresponding intragastric member 500 in its intended formation. Upon complete delivery of the intragastric member 500 into the gastric lumen, the proximal stopper 725 is engaged with a proximal end of the intragastric member 500 to further secure the intragastric member 500 in its intended formation.

The cinching member 510 is tied or otherwise secured to the distal stopper 724 and the proximal stopper 725 so as to form a complete intragastric member 500 as shown in FIG. 45. The size and diameter of the distal stopper 724 and the proximal stopper 725 can vary depending on the size of the intragastric member 500. The embodiment illustrated in FIG. 45 includes two stoppers 724, 725 to secure the intragastric member 500 in its formation. However, alternative embodiments can include varying configurations. For example, one configuration can include a distal stopper 724 engaged to a distal end of the intragastric member 500 and a generic rubber stopper or other similar securing device engaged to a proximal end of the intragastric member 500.

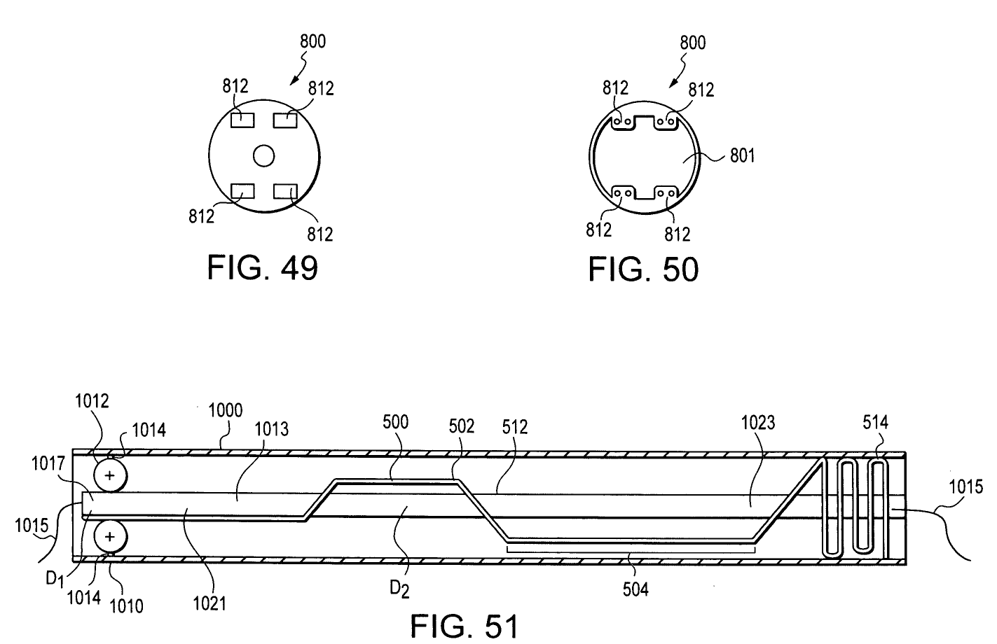

FIGS. 46-47 depict a delivery system 900 in which the intragastric member 500 is delivered through an overtube 800 having a plurality of rollers 810 positioned along the lumen 806 of the overtube 800. The rollers 810 can be positioned along at least one end of the overtube 800. In this embodiment, a set of rollers 810 are positioned along a distal end 802 of the overtube 800 (see FIG. 48). In addition, rollers 810 can be positioned on a proximal end 804 of the overtube 800. The rollers 810 can rotate in a clockwise or counter clockwise direction to facilitate the intragastric member 500 as it passes along the lumen 806 during delivery. The overtube 800 can further include a plurality of grooves 812 along the lumen 806 of the overtube 800 (see FIGS. 49-50). In the illustrative embodiments, four grooves 812 (see FIG. 49) are located along the inside of the overtube 800, however, a skilled artisan would appreciate that other designs could include a smaller or larger number of grooves 812. Each groove 812 can be engaged with an end of the roller 810 that extends along the lumen 806 of the overtube 800. Of course, the overtube 800 could comprise a different number of rollers 810, or the rollers 810 could be eliminated entirely. The grooves 812 can also provide housing for a cable system used to pass the intragastric member 500 along the lumen 806 of the overtube 800.

After the intragastric member 500 is loaded into the overtube 800, the groove 812 and roller 810 assembly operate in conjunction to pass the intragastric member 500 from the proximal end 804 to the distal end 802 of the overtube 800. Once the intragastric member 500 has been introduced into the gastric lumen, the cinching member 510 is pulled back through the overtube 800, thereby releasing the intragastric member 500 into the gastric lumen.

FIG. 51 depicts another embodiment of a delivery system of the present invention. In this embodiment, the separate bundles 514 of the intragastric member 500 are inserted into the gastric lumen one at a time by loading the intragastric member 500 into an overtube 1000 comprising mechanically driven rollers 1010, 1012 and delivering it through the esophagus and into the gastric lumen where the strips 502 are gathered in a butterfly or bow-tie formation. The rollers 1010, 1012 are attached to the end of the overtube 1000 utilizing a connection mechanism such as brackets 1014, posts or other suitable method within the scope of the invention. In addition, the rollers 1010, 1012 are connectable to an electric motor or similar means of power to cause and control rotation of the rollers 1010, 1012 to facilitate delivery of the intragastric member 500 into the gastric lumen. In an alternative embodiment, the overtube 1000 can include other mechanisms to pull the intragastric member 500 within the lumen of the patient. For example, FIG. 53 depicts a gear mechanism 1019 comprising a right angled gear component having a plurality of ribs 1021 engaged to the gear mechanism 1019 to facilitate delivery of the intragastric member 500 into the gastric lumen. In another embodiment illustrated in FIG. 54, the overtube 1000 can include a chain 1031 and pulley arrangement to deliver the intragastric member 500 into the gastric lumen, wherein the chain 1031 rotates about and around a crank 1032 to facilitate delivery. Similar to the rollers 1010, 1012, these alternative devices are mechanically attached to the overtube 1000 or similar delivery device.

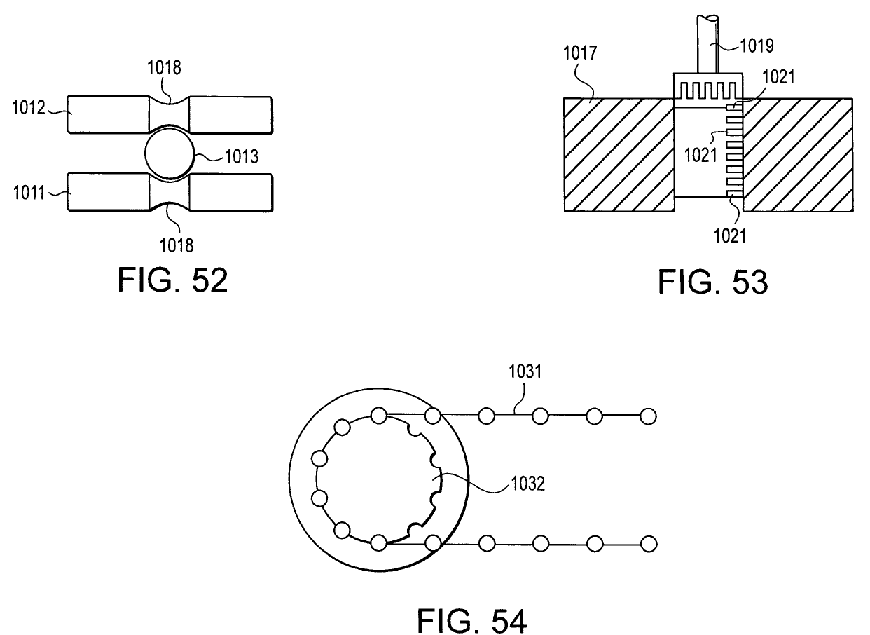

Referring to FIG. 51, the intragastric member 500 is loaded into the overtube 1000 by threading an inner delivery tube 1013 through the apertures 512 of each strip 502. The inner delivery tube 1013 comprises a proximal end 1023 and a distal end 1021 wherein the distal end 1021 comprises a distal tip 1017. Each of the rollers 1010, 1012 comprise a groove 1018 extending about and around the surface of the roller 1010, 1012 that compliments the shape of the inner delivery tube 1013 (FIG. 52). The grooves 1018 are recessed so as to provide an increase in surface area between the mechanically driven rollers 1010, 1012 (FIG. 52) and the inner delivery tube 1013, so as to engage the strip 502 therebetween. The rollers 1010, 1012 are mechanically driven and provide tension sufficient to pull the intragastric member 500 along the overtube 1000 during delivery into the gastric lumen. The overtube 1000 further includes a cinching member 1015, such as nylon thread or similar thread-like structure, which passes through a main lumen of the inner delivery tube 1013 wherein it can be released upon delivery into the gastric lumen.

Delivery of the intragastric member 500 begins by inserting the distal end 1021 of the overtube 1000 in the gastric lumen of the patient while the proximal end 1023 will remain external to the patient. The rollers 1010, 1012 of the overtube 1000 facilitate the delivery of each bundle 514 from the overtube 1000 into the gastric lumen. Once the distal end 1021 of the overtube 1000 is positioned in the gastric lumen, the first bundle 514 is threaded over the proximal (rearward) end 1023 of the inner delivery tube 1013 by passing the apertures 512 over the inner delivery tube 1013. Utilizing the rollers 1010, 1012, each bundle 514 is pulled from the proximal end 1023 of the overtube 1000, and pulled towards the distal end 1021 of the overtube 1000 so as to pull the folds 504 of the first bundle 514 past the distal end 1021 of the overtube 1000.

As shown in FIG. 51, the distal tip 1017 of the inner delivery tube 1013 comprises a first diameter D1, wherein the remainder of the inner delivery tube 1013 comprises a second diameter D2. The first diameter D1 is sized and configured to facilitate delivery of the intragastric member 500 as it passes across the rollers 1010, 1012. The second diameter D2 of the inner delivery tube 1013 is sized and configured to align with the apertures 512 of the strips 502 after each bundle 514 transitions along the length of the overtube 1000 during delivery. As the inner delivery tube 1013 is withdrawn, the subsequent bundle 514 is pulled past the proximal end 1023 of the inner delivery tube 1013 onto the distal tip 1017. A skilled artisan would appreciate that other designs comprising an inner delivery tube of different diameters could be utilized.

This procedure is then repeated until all of the bundles 514 have been inserted into the gastric lumen. The bundles 514 are then secured together by pushing a proximal stopper or similar device along the outer delivery tube 518 so as to press against the last bundle 514 to be inserted. The inner delivery tube 1013 is then withdrawn so as to leave the cinching member 510 extending through the apertures 512 of all of the bundles 514. The cinching member 510 is then tied or otherwise secured to the second stopper 524 so as to form a complete intragastric member 500.

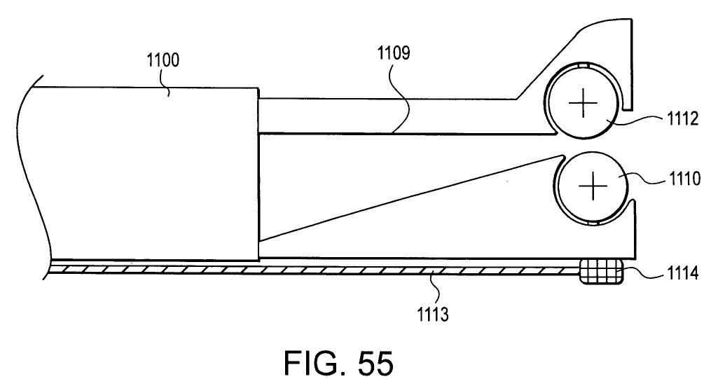

FIG. 55 depicts another embodiment of a delivery system of the present invention. In this embodiment, the overtube 1100 comprises rollers 1110, 1112 engaged along an inner surface 1109 along a distal end of the overtube 1100. During delivery of the intragastric member 500, the rollers 1110, 1112 engage the intragastric member 500 and pull the intragastric member 500 into the gastric lumen of the patient. A flexible shaft 1113 comprising a gearbox 1114 is engaged along the body of the overtube 1100 to support the overtube 1100. The gearbox 1114 is connected to the rollers 1110, 1112 to regulate the rotation of the rollers 1110, 1112 during deliver of the intragastric member 500 into the gastric lumen of the patient.

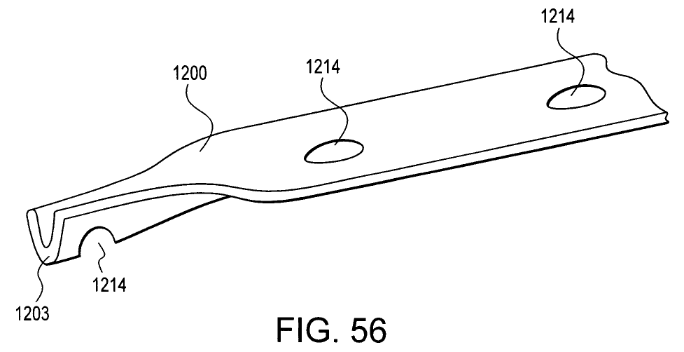

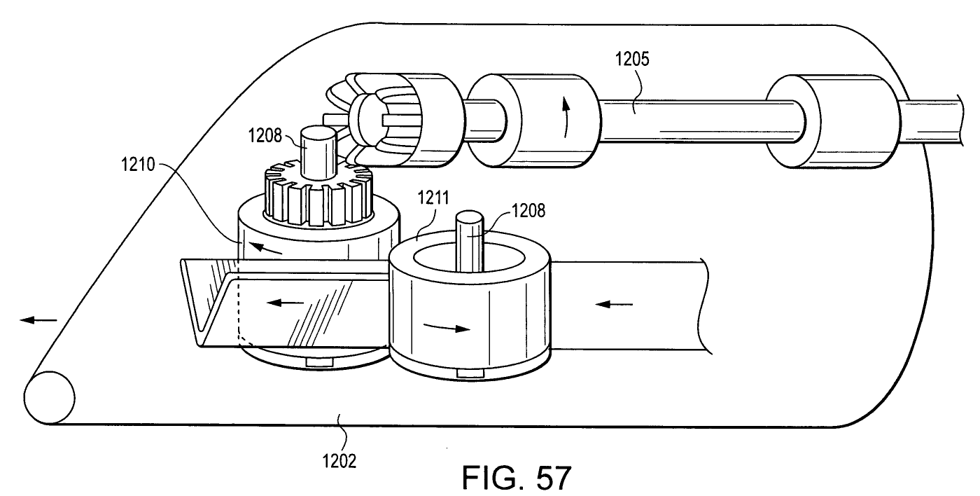

FIGS. 56-57 depict another embodiment of an intragastric member 1200 of the present invention. In this embodiment, the intragastric member 1200 comprises a single strip of high-density polyethylene 1202 that comprises a folded edge 1203 along a distal end of the intragastric member 1200. The intragastric member 1200 further comprises a plurality of apertures 1214 spaced along the intragastric member 1200 that allow the intragastric member 1200 to be threaded onto a delivery mechanism and pulled together upon delivery into the gastric lumen. The folded edge 1203 of the intragastric member 1200 is received by a gear driven roller mechanism 1205. The gear driven roller mechanism 1205 is attached to an overtube to assist in delivery of the intragastric member 1200. The gear driven roller mechanism 1205 comprises a pair of rollers 1210, 1211 that engage the folded edge 1203 of the intragastric member 1200 and thereby pulls the intragastric member 1200 through the overtube 1202 and into the gastric lumen of the patient. The rollers 1210, 1211 are attached to the overtube 1202 with a pair of brackets 1208 engaged to the body of the overtube 1202. The rollers 1210, 1211 can also be attached to utilizing other suitable mechanisms, such as screws and the like, depending on the particular design.

Any other undisclosed or incidental details of the construction or composition of the various elements of the disclosed embodiment of the present invention are not believed to be critical to the achievement of the advantages of the present invention, so long as the elements possess the attributes needed for them to perform as disclosed. The selection of these and other details of construction are believed to be well within the ability of one of even rudimentary skills in this area, in view of the present disclosure. Illustrative embodiments of the present invention have been described in considerable detail for the purpose of disclosing a practical, operative structure whereby the invention may be practiced advantageously. The designs described herein are intended to be exemplary only. T152he novel characteristics of the invention may be incorporated in other structural forms without departing from the spirit and scope of the invention.

1. An intragastric device for the treatment of obesity, the intragastric device comprising:
an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, wherein said intragastric member comprises a plurality of spaced apart openings;
an elongate member having a proximal end and a distal end, wherein the elongate member is threaded through the openings of the intragastric member, and the intragastric member is disposed between the proximal end and distal end of the elongate member when in the second configuration;
a distal stopper engaged to the distal end of the elongate member for securing the intragastric member along the elongate member;
a proximal stopper engaged to the proximal end of the elongate member for locking the intragastric member along the elongate member; and
a cinching member for moving the proximal stopper along the elongate member towards the distal stopper, the cinching member being engaged to the proximal stopper.

2. The intragastric device according to claim 1 further comprising a connector engaged to the proximal end of the elongate member wherein the connector comprises a lumen configured to receive the cinching member as it passes from the proximal stopper.

3. The intragastric device according to claim 3 wherein the proximal stopper comprises a lumen configured to receive the cinching member as it passes from the connector.

4. The intragastric device according to claim 1 further comprising a second distal stopper engaged to the distal stopper to secure the intragastric member upon delivery into the gastric lumen.

5. The intragastric device according to claim 1 further comprising a second proximal stopper engaged to the proximal stopper to secure the intragastric member upon delivery into the gastric lumen.

6. The intragastric device according to claim 5 wherein the cinching member is engaged to the second proximal stopper to secure the intragastric member upon delivery into the gastric lumen.

7. The intragastric device according to claim 1 wherein the elongate member comprises a plurality of ribs disposed between the proximal end and the distal end of the elongate member, the ribs preventing proximal movement of the proximal stopper relative to the elongate member.

8. The intragastric device of claim 1 wherein the cinching member is attached to the distal stopper, the distal stopper comprising a lumen to receive the cinching member as it passes through a lumen of the elongate member.

9. The intragastric device according to claim 8 further comprising a locking member disposed along the distal end of the elongate member wherein the locking member comprises a lumen to receive the cinching member as it passes through the lumen of the elongate member.

10. The intragastric device of claim 8 wherein the proximal stopper comprises a lumen to receive the cinching member as it passes through the lumen of the elongate member.

11. The intragastric device according to claim 9 wherein when the intragastric member is secured, the distal stopper is engaged to the locking member by pulling the cinching member proximally to remove the cinching member from the lumen of the elongate member.

12. The intragastric device according to claim 9 wherein when the intragastric member is secured, the proximal stopper is engaged to the elongate member by pulling the cinching member proximally to remove the cinching member from the lumen of the elongate member.

13. The intragastric device according to claim 1 wherein the intragastric member comprises a plurality of openings extending along the surface of the intragastric member to reduce the mass of the intragastric member.

14. The intragastric device according to claim 1 wherein the intragastric member comprises a folded edge, wherein the folded edge engages a roller mechanism to facilitate the delivery of the intragastric member into the gastric lumen.

15. The intragastric device according to claim 1 wherein the intragastric member comprises a continuous strip of material that has been folded to form a plurality of loops, said plurality of loops being connected together to form a shape suggestive of a butterfly or bow-tie in the second configuration.

16. An intragastric device for the treatment of obesity, the intragastric device comprising:
an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, wherein said intragastric member comprises a plurality of spaced apart openings;
a cinching member having a proximal end and a distal end, wherein the cinching member is threaded through the openings of the intragastric member, the intragastric member being disposed between the proximal end and the distal end of the cinching member;
a distal stopper engaged to the distal end of the cinching member for securing the intragastric member along the cinching member; and
a proximal stopper engaged to the proximal end of the cinching member for locking the intragastric member along the cinching member.

17. The intragastric device according to claim 16 wherein the distal stopper comprises at least one lumen to receive the cinching member.

18. The intragastric device of claim 16 wherein the proximal stopper comprises at least one lumen for receiving the cinching member.

19. The intragastric device of claim 16 further comprising a second proximal stopper comprising at least one lumen configured for receiving the cinching member.

20. The intragastric device according to claim 19 wherein when the intragastric member is secured, the second proximal stopper is engaged to the proximal stopper by pulling the cinching member proximally through the at least one lumen of the second proximal stopper.

21. The intragastric device according to claim 19 wherein the second proximal stopper comprises a proximal end and a distal end, wherein the proximal end is engaged to a second cinching member and the distal end is engaged to the cinching member.

22. The intragastric device according to claim 16 wherein the distal stopper comprises at least one lumen for securing a wire guide.

23. The intragastric device according to claim 18 wherein the at least one lumen of the proximal stopper comprises a first lumen having a first diameter and a second lumen having a second diameter, wherein the first diameter is smaller than the second diameter for securing the second proximal stopper in the second lumen of the proximal stopper.

24. The intragastric device according to claim 23 wherein when the intragastric member is secured, the first diameter of the lumen allows the second proximal stopper to pass distally through the lumen and prevents the second proximal stopper from passing proximally through the lumen.

25. The intragastric device according to claim 16 wherein the intragastric member comprises a continuous strip of material that has been folded to form a plurality of loops, said plurality of loops being connected together to form a shape suggestive of a butterfly or bow-tie in the second configuration.

26. An intragastric device for the treatment of obesity, the intragastric device comprising:
an intragastric member that is re-configurable from a first configuration to a second configuration, the first configuration being sufficiently small to permit introduction of said intragastric member into a gastric lumen of a mammal, the second configuration being sufficiently large to prevent said intragastric device from passing through the mammal's pylorus, the intragastric member comprising a plurality of spaced apart openings;
an outer delivery tube having a main lumen, a proximal end, and a distal end, wherein the intragastric member is loaded between the proximal end and distal end of the outer delivery tube in the first configuration, the intragastric member being loaded by passing the outer delivery tube through the openings of the intragastric member;
an inner delivery tube engaged with the main lumen of the outer delivery tube; and
at least one stopper engaged with the inner delivery tube to secure the intragastric member upon delivery into the gastric lumen.

27. The intragastric device according to claim 26 further comprising an overtube having a proximal end, a distal end, and a lumen configured to receive the intragastric member in the first configuration for delivery into the gastric lumen, the outer delivery tube being movably disposed within the lumen of the overtube.

28. The intragastric device according to claim 27 wherein the overtube comprises a plurality of rollers to facilitate delivery of the intragastric member into the gastric lumen.

29. The intragastric device according to claim 26 wherein the inner delivery tube comprises a distal section having a reduced diameter, the reduced diameter being configured to prevent proximal movement of the intragastric member.

30. The intragastric device according to claim 26 wherein the outer delivery tube comprises a flared distal end for preventing proximal movement of the intragastric member.

31. The intragastric device according to claim 26 wherein the intragastric member comprises a continuous strip of material that has been folded to form a plurality of loops, said plurality of loops being connected together to form a shape suggestive of a butterfly or bow-tie in the second configuration.
